(19) United States
(12) Patent Application Publication
(10) Pub. No.: US 2024/0423979 A1
(43) Pub. Date: Dec. 26, 2024

(54) THERAPEUTICALLY EFFECTIVE COMBINATION OF A FLT3 INHIBITOR AND A BCL-2 INHIBITOR FOR THE TREATMENT OF ACUTE MYELOID LEUKEMIA

(71) Applicant: Hanmi Pharm. Co., Ltd., Hwaseong-si, Gyeonggi-Do (KR)

(72) Inventors: Ji Sook KIM, Hwaseongi-Si (KR); Jae Yul CHOI, Hwaseong-si (KR); Young Gil AHN, Hwaseong-si (KR)

(21) Appl. No.: 18/703,219

(22) PCT Filed: Oct. 20, 2022
(86) PCT No.: PCT/KR2022/016095

§ 371 Date: Apr. 19, 2024

(30) Foreign Application Priority Data
Oct. 20, 2021 (KR) 10-2021-0140237

(51) Int. Cl. A61K 31/506; A61K 31/635; A61K 31/706; A61P 35/02

(52) U.S. Cl. CPC A61K 31/506; A61K 31/635; A61K 31/706; A61P 35/02

(57) ABSTRACT

This invention relates to pharmaceutical compositions, pharmaceutical combinations and methods for the treatment of acute myeloid leukemia by combined use of a therapeutically effective combination of a compound of Chemical Formula 1, or a pharmaceutically acceptable salt thereof, solvate thereof, stereoisomer thereof, tautomer thereof, or combination thereof, wherein Ea, Eb, Ec, Ed, Z′, X′, Q, and k are defined herein; and a Bcl-2 inhibitor, or a Bcl-2 inhibitor and a hypomethylating agent.

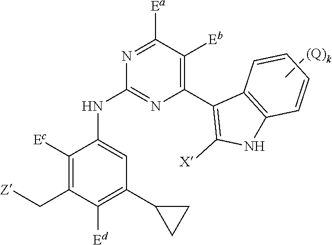

### TECHNICAL FIELD

### Cross-Reference to Related Applications

The present application claims priority to, and the benefit of Korean Patent Application No. 10-2021-0140237, filed on Oct. 20, 2021, the contents of which is hereby incorporated by reference in its entirety for all purposes.

### TECHNICAL FIELD

It relates to a pharmaceutical composition for the treatment of acute myeloid leukemia, comprising a therapeutically effective combination of an FLT3 inhibitor and a Bcl-2 inhibitor, and more specifically, to a pharmaceutical composition for the treatment of acute myeloid leukemia, including FLT3 inhibitors administered in combination with Bcl-2 inhibitors or with Bcl-2 inhibitors and hypomethylating agents.

### BACKGROUND ART

Fms-like tyrosine kinase-3 (FLT3) is one of the most frequently mutated genes in acute myeloid leukemia (AML). Mutant FLT3 (Mutant FLT3) is a mutation expressed in leukemia cells appearing in subpopulation of patients with acute myeloid leukemia (AML). Activating mutations in FLT3, such as Internal Tandem Duplication (ITD) in the proximal domain, account for about 25-30% of newly diagnosed AML cases and are associated with poor prognosis. (British Journal of Hematology, 2003, 122, 523-538). It is known that FLT3 mutations occur in about ⅓ of patients with acute myeloid leukemia (AML). In addition, although there are several clinically available FLT3 inhibitors, drug-resistant leukocyte cells were observed in AML patients treated with these FLT3 inhibitors, indicating drug resistance (Cancer Science 2020 Volume 111:312-322). Additionally, conventional Acute Myeloid Leukemia (AML) standard chemotherapy cannot target AML stem/progenitor cells, which frequently causes disease recurrence in patients, thereby limiting long-term efficacy (Oncogene 2010 Volume 29: 5120-5134). Therefore, there is a need for compositions, combinations and methods that can effectively treat patients with mutated acute leukemia.

### DISCLOSURE

In embodiments, the present disclosure provides pharmaceutical compositions, combinations, and methods for the treatment of cancer e.g., acute myeloid leukemia by the combined use of a FLT3 inhibitor, or a pharmaceutically acceptable salt thereof, stereoisomer thereof, solvate thereof or combination thereof and (i) at least one B-cell lymphoma-2 (Bcl-2) inhibitor or (ii) at least one Bcl-2 inhibitor and at least one hypomethylating agent (HMA).

In one aspect, provided herein is a pharmaceutical composition for the treatment of acute myeloid leukemia, comprising a combination of FLT3 inhibitors and Bcl-2 inhibitors. In embodiments, the present disclosure provides a pharmaceutical composition containing FLT3 inhibitors, which are administered in combination with a Bcl-2 inhibitor or with a Bcl-2 inhibitor and a hypomethylating agent.

In another aspect, the present disclosure provides a pharmaceutical composition for the treatment of acute myeloid leukemia which is composed of a therapeutically effective combination of a Bcl-2 inhibitor and a FLT3 inhibitor. In embodiments, the present disclosure provides a pharmaceutical composition comprising a Bcl-2 inhibitor administered as a combination with a FLT3 inhibitor, or as a combination with a FLT3 inhibitor and a hypomethylating agent.

Yet another aspect is to provide a method for treating acute myeloid leukemia using the pharmaceutical composition described above.

An aspect of the present disclosure is to provide a composition comprising an Fms-like tyrosine kinase-3 (FLT3) inhibitor, wherein the FLT3 inhibitor is selected from a compound of Chemical Formula 1 below, stereoisomers, catabolic isomers, and combinations thereof.

In embodiments, the present disclosure provides a pharmaceutical combination comprising a therapeutically effective amount of a compound of Chemical Formula 1, or a pharmaceutically acceptable salt thereof, solvate thereof, stereoisomer thereof, tautomer thereof, or combination thereof and (i) a B-cell lymphoma-2 (Bcl-2) inhibitor or (ii) Bcl-2 inhibitor and a hypomethylating agent (HMA);

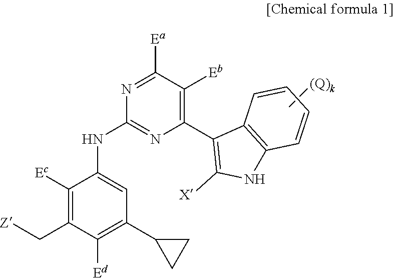

wherein:

E^{a} is hydrogen, hydroxy or C1-4 alkoxy;

E^{b} is hydrogen, halogen, C1-4 alkyl or C1-4 fluoroalkyl;

E^{c} and E^{d} are hydrogen or hydroxy independently of each other;

X′ is hydrogen or hydroxy;

k is an integer from 1 to 2;

each Q is a hydroxy, halogen, C1-4 alkyl, hydroxy C1-4 alkyl or C1-4 alkoxy independently of each other;

Z′ is a monovalent functional group shown in Chemical Formula 2;

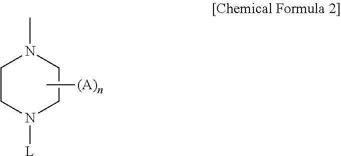

wherein, in Chemical Formula 2,

each A is a functional group independently selected from hydroxy, C1-4 alkyl, and hydroxy C1-4 alkyl, where at least one A is C1-4 alkyl;

n is an integer from 1 to 2; and

L is hydrogen, C1-4 alkyl, hydroxy or hydroxy C1-4 alkyl.

In embodiments, a pharmaceutical combination is provided comprising a therapeutically effective amount of a compound of Chemical Formula 3, or a pharmaceutically acceptable salt thereof, solvate thereof, stereoisomer thereof, tautomer thereof, or combination thereof and (i) a B-cell lymphoma-2 (Bcl-2) inhibitor or (ii) Bcl-2 inhibitor and a hypomethylating agent (HMA);

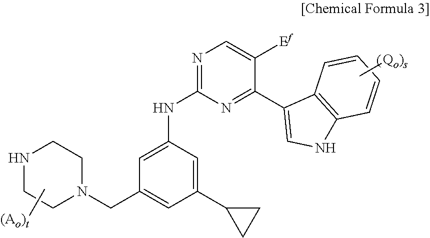

wherein:

E^{f} is fluorine, chlorine, bromine or iodine;

Q^{o} is hydroxy, halogen, C1-4 alkyl, hydroxy C1-4 alkyl or C1-4 alkoxy;

s is an integer from 1 to 2;

A^{o} is a functional group selected from hydroxy, C1-4 alkyl and hydroxy C1-4 alkyl; and

t is an integer from 1 to 2.

In embodiments, a pharmaceutical combination is provided comprising a therapeutically effective amount of the compound:

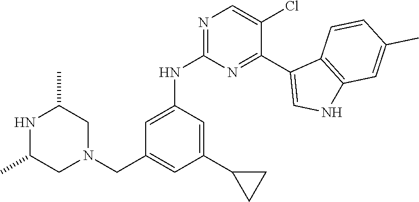

or a pharmaceutically acceptable salt thereof, solvate thereof, stereoisomer thereof, tautomer thereof, or combination thereof and (i) a B-cell lymphoma-2 (Bcl-2) inhibitor or (ii) Bcl-2 inhibitor and a hypomethylating agent (HMA).

In embodiments, a pharmaceutical combination is provided comprising a therapeutically effective amount of the compound

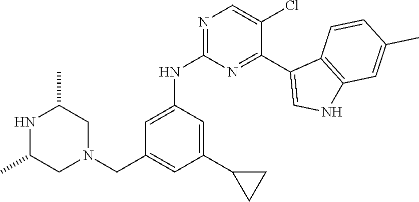

or a pharmaceutically acceptable salt thereof or solvate thereof and (i) a B-cell lymphoma-2 (Bcl-2) inhibitor or (ii) Bcl-2 inhibitor and a hypomethylating agent (HMA).

In one embodiment, a pharmaceutical combination is provided comprising a compound of the present disclosure (e.g., a compound of chemical Formula 1 or chemical Formula 3) and a Bcl-2 inhibitor or a Bcl-2 inhibitor and a hypomethylating agent (HMA), in a single dosage form or in separate dosage forms. In another embodiment, where the pharmaceutical combination comprising a compound of the present disclosure (e.g., a compound of chemical Formula 1 or chemical Formula 3) and a Bcl-2 inhibitor or a Bcl-2 inhibitor and a hypomethylating agent (HMA), are in separate dosage forms and are administered by the same mode of administration or a different mode of administration. In one embodiment, the separate dosage forms of a pharmaceutical combination provided herein, are co-administered by simultaneous administration, sequential administration, overlapping administration, interval administration, continuous administration, or a combination thereof.

In embodiments, the present disclosure provides a method of treating cancer (e.g., acute myeloid leukemia) in a subject in need thereof, comprising administering to the subject a compound of the present disclosure (e.g., a compound of chemical Formula 1 or Formula 3) and a Bcl-2 inhibitor or a Bcl-2 inhibitor and a hypomethylating agent (HMA).

The composition provides a pharmaceutical composition for the treatment of acute myeloid leukemia, characterized in that it is administered in combination with a B-cell lymphoma-2 (Bcl-2) inhibitor or in combination with a Bcl-2 inhibitor and a hypomethylating agent (HMA).

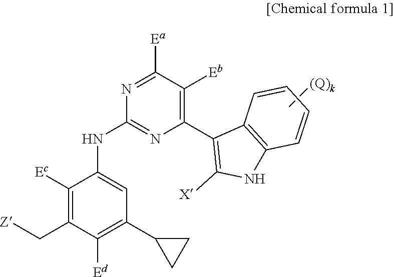

In Chemical Formula 1 above,

Ea is hydrogen, hydroxy or C1-4 alkoxy;

Eb is hydrogen, halogen, C1-4 alkyl or C1-4 fluoroalkyl;

Ec and Ed are hydrogen or hydroxy independently of each other;

X′ is hydrogen or hydroxy;

k is an integer from 1 to 2;

Each Q is a hydroxy, halogen, C1-4 alkyl, hydroxy C1-4 alkyl or C1-4 alkoxy independently of each other;

Z′ is a monovalent functional group shown in Chemical Formula 2;

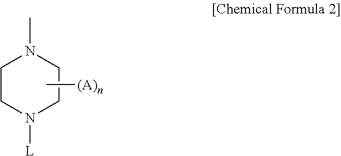

In this case, in Chemical Formula 2,

Each A is a functional group independently selected from hydroxy, C1-4 alkyl, and hydroxy C1-4 alkyl, where at least one A is C1-4 alkyl;

n is an integer from 1 to 2;

L is hydrogen, C1-4 alkyl, hydroxy or hydroxy C1-4 alkyl.

Another aspect is a composition containing a Bcl-2 inhibitor, and the composition provides a pharmaceutical composition for the treatment of acute myeloid leukemia, characterized in that it is co-administered with a compound selected from 5-chloro-N-(3-cyclopropyl-5-(((3R,5S)-3,5-dimethylpiperazine-1-day) methyl) phenyl)-4-(6-methyl-1H-indol-3-days) pyrimidin-2-amine, stereoisomers, tautomers, and combinations thereof, or; a compound selected from 5-chloro-N-(3-cyclopropyl-5-(((3R,5S)-3,5-dimethylpiperazin-1-day) methyl) phenyl)-4-(6-methyl-1H-Indol-3-days) pyrimidin-2-amine, a stereoisomer, tautomer, and combinations thereof, and hypomethylating agent.

Yet another aspect is to provide a method got treating acute myeloid leukemia using the pharmaceutical composition described above.

The pharmaceutical compositions and treatment methods according to the aspects can increase the treatment effect for acute myeloid leukemia, show excellent treatment effects in patients with acute myeloid leukemia with FLT3 mutation, and can also show effects for the treatment of other malignant tumors in the blood system.

The combination therapy of FLT3 inhibitor and Bcl-2 inhibitor using the above pharmaceutical composition, or the combination therapy of FLT3 inhibitor, Bcl-2 inhibitor, and hypomethylating agent, respectively, has improved treatment effects compared to the effects when each is administered alone. This therapeutic effect may represent a synergistic therapeutic effect greater than the arithmetic sum of the combination of two or more drugs.

### BRIEF DESCRIPTION OF DRAWINGS

FIG. 1 shows the anti-tumor effect when compound A and venetoclax are administered in combination to nude mice in which MV-4-11 cell lines are transplanted. The Y-axis represents the tumor volume (MM3) of the surviving mouse in each experimental group, and the X-axis represents the number of days of administration. (**p<0.01, ***p<0.001, and ****p<0.0001 vs. Compound A; #p<0.05, and ###p<0.0001 vs. venetoclax; two-way ANOVA)

FIG. 2 Shows the anti-tumor effect when compound A and venetoclax are administered in combination to nude mice in which MOLM-13 cell lines are transplanted. The Y-axis represents the tumor volume (MM3) of the surviving mouse in each experimental group, and X-axis represents the number of days of administration. (##p<0.01 vs. venetoclax; Two-way ANOVA)

FIG. 3 shows a bioluminescent image measured after administering each treatment solution or drug alone or in combination in a NOD/SCID mouse in which the MOLM-14 Luc/GFP cell line is bone marrow allotropically transplanted.

FIG. 4 shows an anti-tumor effect after administering each treatment solution or drug alone or in combination in a NOD/SCID mouse in which the MOLM-14 Luc/GFP cell line is bone marrow allotropically transplanted. The Y-axis represents the log average radiation emitting diagram obtained from the radiation emitting measured from the mouse in each experimental group, and the X-axis represents the number of days of administration. (**p<0.01, ***p<0.001 vs. compound A alone; Dunnett's test after comparing two-way ANOVA)

### MODE FOR THE INVENTION

All technical terms used in the present invention, unless otherwise defined, have the meaning as commonly understood by a person of ordinary skill in the scope of the present invention. Additionally, although preferred methods and samples are described herein, similar or equivalent methods are also included in the scope of the present invention. Also, the numerical values described in this specification are considered to include the meaning of “about” even if not specified. As used herein, the term “about” refers to a value that is within about 10% higher or lower than the stated value. The contents of all publications incorporated herein by reference are hereby incorporated by reference in their entirety.

### 1. Therapy Drugs

### FLT3 Inhibitor

In one specific example, the FLT3 inhibitor can be a compound selected from the compounds of chemical formula 1 below, stereoisomer, tautomers, and a combination thereof.

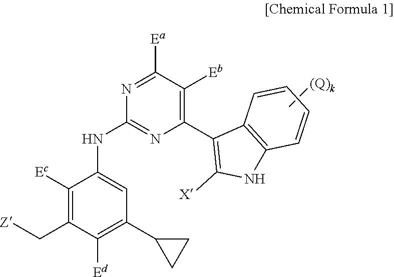

In Chemical Formula 1 above,

Ea is hydrogen, hydroxy or C1-4 alkoxy;

Eb is hydrogen, halogen, C1-4 alkyl or C1-4 fluoroalkyl;

Ec and Ed are hydrogen or hydroxy, independently of each other;

X′ is hydrogen or hydroxy;

k is an integer from 1 to 2;

each Q is hydroxy, halogen, C1-4 alkyl, hydroxy C1-4 alkyl or C1-4 alkoxy, independently of each other;

Z′ is a monovalent functional group represented by Chemical Formula 2;

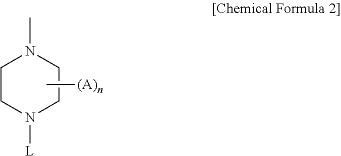

In this case, in Chemical Formula 2 above,

Each A is independently a functional group selected from hydroxy, C1-4 alkyl and hydroxy C1-4 alkyl, wherein at least one A is C1-4 alkyl;

n is an integer from 1 to 2;

L is hydrogen, C1-4 alkyl, hydroxy or hydroxyC1-4 alkyl.

In one specific example, the FLT3 inhibitor may be a compound selected from a compound of Chemical Formula 3 below, stereoisomer, tautomer, and combinations thereof.

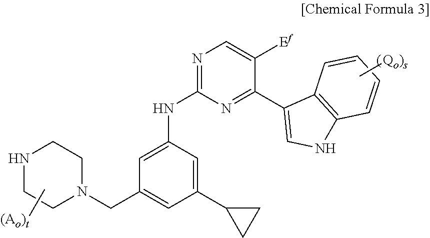

In Chemical Formula 3 above,

Ef is fluorine, chlorine, bromine or iodine;

Qo is hydroxy, halogen, C1-4 alkyl, hydroxy C1-4 alkyl or C1-4 alkoxy;

s is an integer from 1 to 2;

Ao is a functional group selected from hydroxy, C1-4 alkyl and hydroxy C1-4 alkyl;

t is an integer from 1 to 2.

In one specific example, the FLT3 inhibitor may be any one selected from the group consisting of the following compounds.
1) 5-chloro-N-(3-cyclopropyl-5-(((3R,5S)-3,5-dimethylpiperazine-1-yl) methyl) phenyl)-4-(6-fluoro-1H-indol-3-yl) pyrimidine-2-amine
2) 5-chloro-4-(6-chloro-1H-indol-3-yl)-N-(3-cyclopropyl-5-(((3R,5S)-3,5-dimethylpiperazine-1-yl) methyl) phenyl) pyrimidine-2-amine
3) 2-((2R,6S)-4-(3-((5-chloro-4-(6-fluoro-1H-indol-3-yl) pyrimidine-2-yl) amino)-5-cyclopropylbenzyl)-2,6-dimethylpiperazin-1-yl) ethan-1-ol
4) 2-((2R,6S)-4-(3-((5-chloro-4-(1H-indol-3-yl) pyrimidine-2-yl) amino)-5-cyclopropylbenzyl)-2,6-dimethylpiperazin-1-yl) ethan-1-ol
5) 2-((2R,6S)-4-(3-((5-chloro-4-(6-methyl-1H-indol-3-yl) pyrimidine-2-yl) amino)-5-cyclopropylbenzyl)-2,6-dimethylpiperazin-1-yl) ethan-1-ol
6) (R)-5-chloro-N-(3-cyclopropyl-5-((3-methylpiperazine-1-yl) methyl) phenyl)-4-(1H-indol-3-yl) pyrimidine-2-amine
7) (R)-5-chloro-N-(3-cyclopropyl-5-((3-methylpiperazine-1-yl) methyl) phenyl)-4-(6-methyl-1H-indol-3-yl) pyrimidine-2-amine
8) 5-chloro-N-(3-cyclopropyl-5-(((3R,5S)-3,5-dimethylpiperazine-1-yl) methyl) phenyl)-4-(6-methyl-1H-indol-3-yl) pyrimidine 2-amine
9) 5-chloro-N-(3-cyclopropyl-5-(((3S,5R)-3-ethyl-5-methylpiperazine-1-yl) methyl) phenyl)-4-(6-methyl-1H-indol-3-yl) pyrimidine-2-amine
10) 5-chloro-N-(3-cyclopropyl-5-((3,5-dimethylpiperazine-1-yl) methyl) phenyl)-4-(6-methyl-1H-indol-3-yl) pyrimidine-2-amine
11) N-(3-cyclopropyl-5-(((3R,5S)-3,5-dimethylpiperazine-1-yl) methyl) phenyl)-4-(6-methyl-1H-indol-3-yl) pyrimidine-2-amine
12) N-(3-cyclopropyl-5-(((3R,5S)-3,5-dimethylpiperazine-1-yl) methyl) phenyl)-5-fluoro-4-(6-methyl-1H-indol-3-yl) pyrimidine-2-amine
13) N-(3-cyclopropyl-5-(((3R,5S)-3,5-dimethylpiperazine-1-yl) methyl) phenyl)-4-(1H-indol-3-yl)-5-methylpyrimidine-2-amine
14) N-(3-cyclopropyl-5-(((3R,5S)-3,5-dimethylpiperazine-1-yl) methyl) phenyl)-5-methyl-4-(6-methyl-1H-indol-3-yl) pyrimidine-2-amine
15) N-(3-cyclopropyl-5-(((3R,5S)-3,5-dimethylpiperazine-1-yl) methyl) phenyl)-4-(6-methyl-1H-indol-3-yl)-5-(trifluoromethyl) pyrimidine-2-amine
16) (3-(5-chloro-2-((3-cyclopropyl-5-(((3R,5S)-3,5-dimethylpiperazine-1-yl) methyl) phenyl) amino) pyrimidine-4-yl)-1H-indol-6-yl) methanol
17) 5-chloro-N-(3-cyclopropyl-5-(((3R,5S)-3,5-dimethylpiperazine-1-yl) methyl) phenyl)-4-(5-methoxy-6-methyl-1H-indol-3-yl) pyrimidine-2-amine
18) 3-(5-chloro-2-((3-cyclopropyl-5-(((3R,5S)-3,5-dimethylpiperazine-1-yl) methyl) phenyl) amino) pyrimidine-4-yl)-6-methyl-1H-indol-5-ol
19) 3-(5-chloro-2-((3-cyclopropyl-5-(((3R,5S)-3,5-dimethylpiperazine-1-yl) methyl) phenyl) amino) pyrimidine-4-yl)-6-methylindolin-2-one
20) 5-chloro-N-(3-cyclopropyl-5-(((3R,5S)-3,5-dimethylpiperazine-1-yl) methyl) phenyl)-4-methoxy-6-(6-methyl-1H-indol-3-yl) pyrimidine-2-amine
21) 5-chloro-2-((3-cyclopropyl-5-(((3R,5S)-3,5-dimethylpiperazine-1-yl) methyl) phenyl) amino)-6-(6-methyl-1H-indol-3-yl) pyrimidine-4-ol
22) 3-(5-chloro-2-((3-cyclopropyl-5-(((3R,5S)-3,5-dimethylpiperazine-1-yl) methyl) phenyl) amino) pyrimidine-4-yl)-6-methyl-1H-indol-7-ol
23) 2-((5-chloro-4-(6-methyl-1H-indol-3-yl) pyrimidine-2-yl) amino)-4-cyclopropyl-6-(((3R,5S)-3,5-dimethylpiperazin-1-yl) methyl) phenol
24) 4-((5-chloro-4-(6-methyl-1H-indol-3-yl) pyrimidine-2-yl) amino)-2-cyclopropyl-6-(((3R,5S)-3,5-dimethylpiperazine-1-yl) methyl) phenol
25) (R)-5-chloro-N-(3-cyclopropyl-5-((3,3,5-trimethylpiperazine-1-yl) methyl) phenyl)-4-(6-methyl-1H-indol-3-yl) pyrimidine-2-amine
26) ((2R,6R)-4-(3-((5-chloro-4-(6-methyl-1H-indol-3-yl) pyrimidine-2-yl) amino)-5-cyclopropylbenzyl)-6-methylpiperazine-2-yl) methanol
27) (R)-5-chloro-N-(3-cyclopropyl-5-((5-methyl-4,7-diazaspiro [2.5] octane-7-yl) methyl) phenyl)-4-(6-methyl-1H-indol-3-yl) pyrimidine-2-amine
28) 5-chloro-N-(3-cyclopropyl-5-(((3R,5R)-3,5-dimethylpiperazine-1-yl) methyl) phenyl)-4-(6-methyl-1H-indol-3-yl) pyrimidine-2-amine
29) 5-chloro-N-(3-cyclopropyl-5-(((3S,5S)-3,5-dimethylpiperazine-1-yl) methyl) phenyl)-4-(6-methyl-1H-indol-3-yl) pyrimidine-2-amine
30) 5-chloro-N-(3-cyclopropyl-5-(((3R,5S)-3,4,5-trimethylpiperazine-1-yl) methyl) phenyl)-4-(6-methyl-1H-indol-3-yl) pyrimidine-2-amine
31) (2R,6S)-4-(3-((5-chloro-4-(6-methyl-1H-indol-3-yl) pyrimidine-2-yl) amino)-5-cyclo propylbenzyl)-2,6-dimethylpiperazine-1-ol
32) (2R,6S)-4-(3-cyclopropyl-5-((4-(6-methyl-1H-indol-3-yl) pyrimidine-2-yl) amino) benzyl)-2,6-dimethylpiperazine-1-ol.

In one specific example, the FLT3 inhibitor can be 5-chloro-N-(3-cyclopropyl-5-(((3R,5S)-3,5-dimethylpiperazine-1-yl) methyl) phenyl)-4-(6-methyl-1H-indol-3-yl) pyrimidine-2-amine.

As used herein, the term “FTL3 inhibitor” is defined to include a pharmaceutically acceptable salt of the compound(s) above or a solvate thereof. As used herein, “Solvate” includes hydrates.

In embodiments, the compound of Chemical Formula 1 is the FLT3 inhibitor:

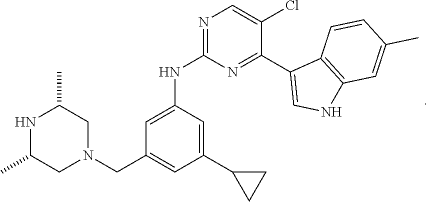

### Bcl-2 Inhibitor

The Bcl-2 protein family is a major regulator of the apoptotic pathway, inter alia the mitochondrial (also so-called “intrinsic”) pathway of apoptosis, which is one of the essential biological processes for maintaining the operational homeostasis of an organism, and it is known to regulate programmed apoptosis triggered by signaling and in response to multiple stress signals. Structural homology domains BH1, BH2, BH3 and BH4 are known to have characteristics in the Bcl-2 family proteins, and the natural expression level of anti-apoptotic Bcl-2 family protein members differs depending on the cell type. For example, the survival of certain cancer cells may be due to dysregulation of the apoptotic pathway caused by overexpression of one or more anti-apoptotic Bcl-2 protein families.

As used herein, a B cell lymphoma-2 (Bcl-2) inhibitor refers to a Bcl-2 protein inhibitor.

Bcl-2 inhibitors can inhibit the survival of overexpressed cancer cells. The Bcl-2 inhibitor, according to an embodiment, may be any substance having properties of suppressing a substance that promotes the survival of the Bcl-2 protein family. For example, the Bcl-2 inhibitor can be venetoclax, navitoclax, obatoclax, oblimersen, SPC-2996, RTA-402, Gossypol, AT-101, obatoclax mesylate, A-371191, A-385358, A-438744, ABT-737, ABT-263, AT-101, BL-11, BL-193, GX-15-003, 2-methoxyantimycin A3, HA-14-1, KF-67544, purpurogallin, TP-TW-37, YC-137 or Z-24.

In one specific example, the Bcl-2 inhibitor may be any one selected from venetoclax, navitoclax, obatoclax, and a combination thereof.

In one specific example, the Bcl-2 inhibitor may be venetoclax.

Venetoclax (or ABT-199/GDC-0199) is a drug referred to by the chemical name of “4-[4-[[2-(4-chlorophenyl)-4,4-dimethylcyclohexen-1-yl] methyl] piperazine-1-yl]-N-[3-nitro-4-(oxan-4-yl methylamino) phenyl] sulfonyl-2-(1H-pyrrolo [2,3-b]pyridin-5-yl oxy) benzamide. It is a Bcl-2 inhibitor approved by the Food and Drug Administration for treatment of chronic lymphocytic leukemia (CLL) and is also known as “Venclexta™”.

Venetoclax, for example, can be formulated in the form of its parent-compound (i.e., as a free base), in the pharmaceutically acceptable salt form of the compound, or in combination with the parent-compound form and pharmaceutically acceptable salt form. Further suitable forms include hydrated or solvated forms of venetoclax. For example, venetoclax may be a crystalline polymorph suitable for incorporation into a pharmaceutical composition further comprising a pharmaceutically acceptable excipient.

The salt and crystalline form of venetoclax is disclosed in U.S. Publication No. 2012/0157470, and its disclosure is included as a reference herein, as its full text is presented. The salt of venetoclax may be prepared during isolation or after purification of the compound.

For example, an acid addition salt of venetoclax is derived from the reaction of venetoclax with an acid. For example, acetate, acid phosphate, adipate, alginate, ascorbate, bicarbonate, citrate, aspartate, benzoate, benzenesulfonate (besylate), bisulfate, bitartrate, butyrate, camphorate, camphorsulfonate, citrate, digluconate, ethanesulfonate, ethanedisulfonate, formate, fumarate, gentisinate, glycerophosphate, gluconate, glucaronate, glutamate, hemisulphate, heptanoate, hexanoate, hydrobromide, hydrochloride, hydroiodide, isonicotinate, 1-hydroxy-2-naphthoate, lactate, lactobionate, maleate, malayate, malonate, mesitylenesulfonate, methanesulfonate, naphthalenesulfonate, nicotinate, nitrate, oxalate, p-toluenesulfonate, pamoate (i.e., 1,1′-methylene-bis-(2-hydroxy-3-naphthoate), pantothenate, pectinate, persulfate, phosphate, picrate, propionate, saccharate, salicylate, succinate, sulfate, tartrate, thiocyanate, trichloroacetate, trifluoroacetate, para-toluenesulfonate and salts including undecanoate salts of venetoclax compounds may be used in the composition of the present invention. A basic side salt including benetoclax and cations such as those derived from the reaction of aluminum, lithium, sodium, potassium, calcium, zinc, and magnesium with bicarbonate, carbonate, hydroxide or phosphate may be used similarly.

Navitoclax is a drug referred to by its chemical name of “4-[4-[[2-(4-chlorophenyl)-5,5-dimethylcyclohexen-1-yl] methyl] piperazin-1-yl]-N-[4-[[(2R)-4-morpholin-4-yl-1-phenylsulfanylbutan-2-yl]amino]-3-(trifluoromethylsulfonyl) phenyl] sulfonylbenzamide”.

Obatoclax is a drug referred to by its chemical name of “2-(2-((3,5-dimethyl-1H-pyrrol-2-yl) methylene)-3-methoxy-2H-pyrrol-5-yl)-1H-indol”.

As used herein, the term “Bcl-2 inhibitor” is defined to include a pharmaceutically acceptable salt of the compound(s) above or a solvate thereof. As used herein, “Solvate” includes hydrates.

### Hypomethylating Agent

In the present specification, the hypomethylating agent refers to a substance or a DNA demethylating agent for hypomethylation of DNA, and is also referred to as a demethylating agent or hypomethylating agent.

DNA methylation is a major mechanism regulating gene expression in cells, and when DNA methylation is increased, the activity of suppressor genes that control cell division and proliferation is blocked, and thus, cell division is not controlled and cancer progresses. For example, hypomethylating agents interfere with DNA methylation to restore tumor suppressor genes, thereby regulating tumor growth, or inhibiting tumor growth by interfering with cellular metabolism with a structure similar to a substance required for tumor cell metabolism.

In one specific example, the hypomethylating agent (HMA) may be any one selected from azacitidine, decitabine, idarubicin, and combinations thereof.

Azacitidine is a drug referred to by the chemical name of “4-amino-1-[(2R,3R,4S,5R)-3,4-dihydroxy-5-(hydroxymethyl)oxolan-2-yl]-1,3,5-triazin-2-one”. Also known as “Vidaza™”, it is also known as a nucleoside metabolism inhibitor (hypomethylating agent) for the treatment of patients with the FAB myelodysplastic syndrome (MDS) subtype.

Decitabine is a drug referred to by the chemical name of “4-amino-1-[(2R,4S,5R)-4-hydroxy-5-(hydroxymethyl) oxolane-2-yl]-1,3,5-triazin-2-one”. Decitabine is used clinically for primary and secondary myelodysplastic syndromes (MDS).

Idarubicin is a drug referred to by the chemical name of “(7S,9S)-9-acetyl-7-[(2R,4S,5S,6S)-4-amino-5-hydroxy-6-methyloxane-2-yl] oxy-6,9,11-trihydroxy-8,10-dihydro-7H-tetracene-5,12-dione”.

As used herein, the term “Hypomethylating agent” is defined to include a pharmaceutically acceptable salt of the compound(s) above or a solvate thereof. As used herein, “Solvate” includes hydrates.

### 2. Therapeutically Effective Drug Combinations

In embodiments, the present disclosure provides a pharmaceutical composition and/or combination comprising a therapeutically effective amount of the compound of the present disclosure (e.g., a compound of Chemical Formula 1 or Chemical Formula 3), as disclosed herein, as the active ingredient, combined with a pharmaceutically acceptable excipient or carrier. The excipients are added to the formulation for a variety of purposes.

In embodiments, the compound of the present disclosure (e.g., a compound of Chemical Formula 1 or Chemical Formula 3), and the Bcl-2 inhibitor or the BCl2-inhibitor and the hypomethylating agent may be formulated into a single pharmaceutical composition comprising a pharmaceutically acceptable excipient or a carrier.

In embodiments, the compound of the present disclosure (e.g., a compound of Chemical Formula 1 or Chemical Formula 3), and the Bcl-2 inhibitor or the BCl2-inhibitor and the hypomethylating agent are formulated into a separate pharmaceutical composition comprising a pharmaceutically acceptable excipient or a carrier.

In embodiments, provided herein is a pharmaceutical combination comprising a therapeutically effective amount of the compound:

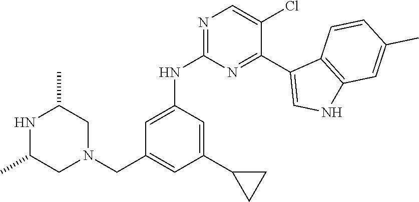

or a pharmaceutically acceptable salt thereof, solvate thereof, stereoisomer thereof, tautomer thereof, or combination thereof, at least one Bcl-2 inhibitor and at least one hypomethylating agent. In embodiments the Bcl-2 inhibitor is venetoclax. In embodiments, the hypomethylating agent is selected from azacitidine, decitabine, and/or idarubicin.

In embodiments, the pharmaceutical composition and/or combination may comprise the compound:

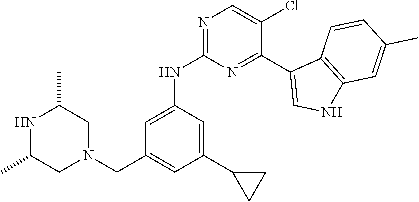

in an amount of about 5 mg to about 500 mg, including about 5 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 250 mg, about 300 mg, about 350 mg, about 400 mg, about 450 mg, or about 500 mg. In embodiments, the pharmaceutical composition and/or combination may comprise about 20 mg, about 40 mg, about 80 mg, about 120 mg, about 160 mg, or about 200 mg.

In embodiments, the present disclosure provides a method for treating acute myeloid leukemia in a subject in need thereof comprising administering a therapeutically effective amount of a composition and/or combination of the present disclosure.

In embodiments, the methods for treating acute myeloid leukemia comprise administering a compound of Formula 1, or Formula 3, or a pharmaceutically acceptable salt thereof, solvate thereof, stereoisomer thereof, tautomer thereof, or combination thereof, as disclosed herein, and at least one Bcl-2 inhibitor.

In embodiments, the methods for treating acute myeloid leukemia comprise administering the compound:

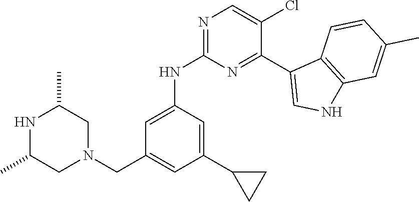

or a pharmaceutically acceptable salt thereof, solvate thereof, stereoisomer thereof, tautomer thereof, or combination thereof, and at least one Bcl-2 inhibitor. In embodiments the Bcl-2 inhibitor is venetoclax.

In embodiments, the methods for treating acute myeloid leukemia comprise administering a compound of Formula 1, or Formula 3, or a pharmaceutically acceptable salt thereof, solvate thereof, stereoisomer thereof, tautomer thereof, or combination thereof, at least one Bcl-2 inhibitor, and at least one hypomethylating agent.

In embodiments, the methods for treating acute myeloid leukemia comprise administering the compound:

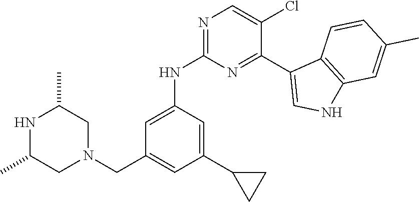

or a pharmaceutically acceptable salt thereof, solvate thereof, stereoisomer thereof, tautomer thereof, or combination thereof, at least one Bcl-2 inhibitor and at least one hypomethylating agent. In embodiments the Bcl-2 inhibitor is venetoclax. In embodiments, the hypomethylating agent is selected from azacitidine, decitabine, and/or idarubicin.

In embodiments of the present disclosure, the venetoclax is administered in an amount of about 80-400 mg daily, including e.g., about 80 mg daily, about 100 mg daily, about 200 mg daily, or about 400 mg daily. In embodiments, venetoclax is administered in an amount of about 100 mg daily on day 1 of a 28-day cycle, about 200 mg daily on day 2 of a 28-day cycle, and about 400 mg daily on days 4-28 of a 28-day cycle.

One aspect of the present disclosure, provides a pharmaceutical composition for the treatment of acute myeloid leukemia comprising a therapeutically effective combination of a FLT3 inhibitor and a Bcl-2 inhibitor.

One aspect is a pharmaceutical composition for the treatment of acute myeloid leukemia comprising a therapeutically effective combination of a FLT3 inhibitor and a Bcl-2 inhibitor, and to provide a pharmaceutical composition comprising a FLT3 inhibitor administered in combination with a Bcl-2 inhibitor or with a Bcl-2 inhibitor and a hypomethylating agent.

In one specific example, the pharmaceutical composition above includes the compound of Chemical Formula 1 as an FLT3 inhibitor, and may be administered in combination with venetoclax.

In one specific example, the pharmaceutical composition above includes the compound of Chemical Formula 3 as an FLT3 inhibitor, and may be administered in combination with venetoclax.

In one specific example, the pharmaceutical composition above comprises 5-chloro-N-(3-cyclopropyl-5-(((3R,5S)-3,5-dimethylpiperazin-1-yl) methyl) phenyl)-4-(6-methyl-1H-indol-3-yl) pyrimidin-2-amine as FLT3 inhibitor, and may be administered in combination with venetoclax.

In one specific example, the pharmaceutical composition above comprises the compound of Chemical Formula 1 as an FLT3 inhibitor, and at least one hypomethylating agent selected from azacitidine, decitabine, and idarubicin; and venetoclax can be administered as a combination.

In one specific example, the pharmaceutical composition above comprises the compound of Chemical Formula 3 as an FLT3 inhibitor, and at least one hypomethylating agent selected from azacitidine, decitabine, and idarubicin; and venetoclax can be administered as a combination.

In one specific example, the pharmaceutical composition above comprises 5-chloro-N-(3-cyclopropyl-5-(((3R,5S)-3,5-dimethylpiperazin-1-yl) methyl) phenyl)-4-(6-methyl-1H-indol-3-yl) pyrimidin-2-amine as a FLT3 inhibitor, and at least one hypomethylating agent selected from azacitidine, decitabine, and idarubicin; and venetoclax can be administered as a combination.

In one specific example, the pharmaceutical composition above comprises 5-chloro-N-(3-cyclopropyl-5-(((3R,5S)-3,5-dimethylpiperazin-1-yl) methyl) phenyl)-4-(6-methyl-1H-indol-3-yl) pyrimidine-2-amine, a pharmaceutically acceptable salt thereof, or a hydrate thereof as a FLT3 inhibitor, and it may be administered in combination with venetoclax and azacitidine.

Another aspect is a pharmaceutical composition for the treatment of acute myeloid leukemia, which includes a therapeutic combination of Bcl-2 inhibitors and FLT3 inhibitors, and provides a pharmaceutical composition comprising a Bcl-2 inhibitor, which is administered in combination with FLT3 inhibitors or with FLT3 inhibitors and hypomethylators.

In one specific example, the pharmaceutical composition above comprises a Bcl-2 inhibitor, and is administered in combination with a compound selected from 5-Chloro-N-(3-cyclopropyl-5-(((3R,5S)-3,5-dimethylpiperazin-1-yl) methyl) phenyl)-4-(6-methyl-1H-indol-3-yl) pyrimidin-2-amine, its stereoisomers, tautomers, and combinations thereof, or with hypomethylating agent and a compound selected from 5-Chloro-N-(3-cyclopropyl-5-(((3R,5S)-3,5-dimethylpiperazin-1-yl) methyl) phenyl)-4-(6-methyl-1H-indol-3-yl) pyrimidin-2-amine, its stereoisomers, tautomers, and combinations thereof.

### 3. Combined Administration Order, Dosage, Formulation

In the pharmaceutical composition and/or pharmaceutical combination according to one specific example, FLT3 inhibitors and Bcl-2 inhibitors, or FLT3 inhibitors, Bcl-2 inhibitors and hypomethylating agents may be administered simultaneously, sequentially, in reverse order, or individually without any specific time limit.

Treatment drugs or combinations of drugs according to one specific example may be administered in combination at effective treatment intervals. The effective interval of treatment is a time period that starts when one of the compounds is administered to the patient and ends at the limit of administration of other compounds where the benefits of combined administration of the two compounds are maintained. Therefore, the combined administration may be simultaneous, sequential, or in any order.

The period of time or cycle of combination administration may 1 week, 28 days, 1 month, 2 months, 3 months, or 4 months, or more in total. The individual drugs may each be administered daily for the entire duration or only a portion of a period or cycle. Alternatively, when 2 or more treatment agents are administered sequentially, each drug may be administered as 2 individual administrations separated by a “specific period of time”. The specific period may be, for example, any period from 1 hour to 15 days. Or, for example, one of the administered drugs may be administered within about 15, 14, 13, 12, 11, 10, 9, 8, 7, 6, 5, 4, 3, 2, or 1 day, or within 24, 23, 22, 21, 20, 19, 18, 17, 16, 15, 14, 13, 12, 11, 10, 9, 8, 7, 6, 5, 4, 3, 2, or 1 hour from the administration of another drug. The period according to the combined administration may be the same or different for each combined drug.

In embodiments, the treatment drug (e.g., FLT3 inhibitor, or compound of Chemical Formula 1 or Chemical Formula 3) is administered as an oral once-daily dose in a 28-day cycle.

In another embodiment, the dosage amount of the treatment drug or compound of Chemical Formula 1 ranges between about 10 mg to about 300 mg. In another embodiment, the dosage amount ranges between about 20 mg to about 240 mg. In another embodiment, the dosage amount ranges in between about 40 mg and about 200 mg. In another embodiment, the dosage amount ranges between about 80 mg, and about 160 mg, or any ranges or subranges therein or in between.

In a specific embodiment, the dosage amount is about 20 mg, 30 mg, 40 mg, 50 mg, 60 mg, 70 mg, 80 mg, 90 mg, 100 mg, 120 mg, 140 mg, 160 mg, 180 mg, 200 mg, 220 mg, 240 mg, 260 mg, 280 mg, and 300 mg.

In a specific embodiment, the dosage amounts are administered to a patient once a day, twice a day, three times a day, or four times a day.

In another embodiment, the dosing will be administered in one week cycles, 2 week cycles, three week cycles, 4 week cycles, 5 week cycles, 6 week cycles, 7 week cycles or 8 week cycles.

In another embodiment, a Bcl-2 inhibitor such as venetoclax is co-administered at a dosage amount ranges from about 80 mg to about 500 mg. In another embodiment, the dosage amount is about 100 mg to about 400 mg. In another embodiment, the dosage amount is 200 mg to about 400 mg. In another embodiment, the dose amount is about 50 mg, 100 mg, 150 mg, 200 mg, 250 mg, 300 mg, 350 mg, 400 mg, 450 mg, or 500 mg.

In a specific embodiment, the Bcl-2 inhibitor is venetoclax.

For example, in one cycle, FLT3 inhibitors are administered daily, while Bcl-2 inhibitors or hypomethylating agents are also administered daily, or can be administered for a partial duration thereof, such as 5 consecutive days, 7 consecutive days, or 10 consecutive days, and 5, 7, and 10 consecutive days may be the first 5, 7, or 10 days of each period or cycle, respectively.

The interval between combination administration of the therapy drug or drug combination may be several seconds, minutes, hours, or days at a predetermined interval, and drug administration may have a pause if necessary.

As used herein, the term “therapeutic effective amount” refers to an amount of a compound administered that is sufficient to prevent the occurrence or alleviate to some extent one or more of the symptoms of the condition or disorder being treated. Additionally, a therapeutic effective amount refers to the amount of treatment that induces a biological or medical response in the tissue system being sought by researchers, veterinarians, doctors, or other clinicians, including relief or partial relief of symptoms of a disease, syndrome, condition, or disorder being treated. The therapeutic effective amount may depend on the recipient of the treatment, the disorder to be treated and its severity, the composition containing the compound, the administration time, the route of administration, the duration of the treatment, the effectiveness of the compound, its clearance rate, and whether another drug is co-administered.

The therapeutic effective amount will be adjusted to the individual requirements of each specific case, including the specific compound to be administered, the route of administration (oral administration, parenteral administration), and the state to be treated as well as the patient to be treated, and may be determined in a known manner in the practice and may vary within a wide tolerance. For example, in the case of oral administration, the daily dose may be about 0.001 to about 100 mg/kg per weight of the patient, for example, about 0.005 to about 30 mg/kg, and for example, about 0.01 to about 10 mg/kg. When administered intravenously, the daily dose may suitably be from about 0.0001 to about 10 mg/kg per weight of the patient, and the entire dose is administered in installments, at least one dose per day. Additionally, mucosal oil preparations are administered at a dose of about 0.001 to about 100 mg/kg per patient weight, and may be administered once a day or several times a day in installments.

The effective amount will be adapted to the individual requirements of each particular case, including the patient being treated as well as the particular compound being administered, the route of administration (oral administration, parenteral administration), and the condition being treated.

A pharmaceutical composition comprising a therapy drug or drug combination according to one circumstance may be provided in a “fixed combination” or in a “non-fixed combination” form.

As used herein, the term “fixed combination” refers to a combination in which an active ingredient, such as the FLT3 inhibitor and the Bcl-2 inhibitor described herein, or the FLT3 inhibitor, the Bcl-2 inhibitor, and the hypomethylating agent, may be simultaneously administered to a patient in the form of a single aggregate.

The term “non-fixed combination” as used herein refers to a combination in which an active ingredient, such as the FLT3 inhibitor and the Bcl-2 inhibitor described herein, or the FLT3 inhibitor, the Bcl-2 inhibitor, and a hypomethylating agent may be administered to a patient simultaneously or sequentially as separate aggregation without a specific time limit.

The FLT3 inhibitors, Bcl-2 inhibitors, and hypomethylating agents described herein include those in which these compound(s) exist as pharmaceutically acceptable salts or solvates thereof.

The term “pharmaceutically acceptable salt” as used herein refers to a salt that is safe and effective in administration to patients and does not adversely affect the treatment quality of the compound. The salt includes an acidic or basic salt present in the compound of the present invention.

Following one specific example, a therapeutic drug or a combination of drugs in the pharmaceutical composition may be provided in the form of a “pharmacologically acceptable salt”, and the formation of the salt may be partial or complete.

As used herein, the term “solvate” is used to describe a molecular complex, which may exist as a compound according to the present invention and one or more pharmaceutically acceptable solvent molecules. It refers to a molecular complex of a compound of the present invention (or a pharmaceutically acceptable salt thereof) with one or more solvent molecules. Such solvent molecules may be those known or commonly used in the pharmaceutical art, for example, water, ethanol, and the like. The term “hydrate” refers to a complex in which the solvent molecule is water.

Following one specific example, the therapeutic drug or drug combination in the pharmaceutical composition may be provided in the form of a “solvate” wherein the solvate includes a hydrate.

Following one specific example, the pharmaceutical composition may further include one or more pharmaceutically acceptable additives. The additives are useful in the preparation of formulations and are any substance known to those skilled in the practice, and may be adjusted as necessary, for example, according to the mode of administration of the drug. For example, the additive may be one or more selected from the group consisting of excipients, binders, disintegrants, lubricants, and any combination thereof.

In one specific example, routes of administration include, but are not limited to, oral, intravenous, intraarterial, intraperitoneal, intradermal, transdermal, intrathecal, intramuscular, intranasal, transmucosal, subcutaneous and rectal administration.

Following one specific example, formulations for administration may be formulated and used in any suitable form according to conventional methods, including oral dosage forms such as tablets, powders, granules, capsules, suspensions, emulsions, syrups, aerosols, etc., external preparations such as ointments and creams, injections, and suppositories and sterile injection solutions, etc.

As used herein, the term “composition” often refers to a pharmaceutical product comprising a therapeutically effective amount of the specified ingredient, as well as any product that results, directly or indirectly, from a combination of the specified ingredients in the specified amounts.

In one specific example, the Bcl-2 inhibitor, or the Bcl-2 inhibitor and the hypomethylating agent administered in combination with the composition comprising the FLT3 inhibitor are respectively administered
(i) concurrently with FLT3 inhibitor;
(ii) sequentially after FLT3 inhibitor is administered first;
(iii) by administering a Bcl-2 inhibitor, or a Bcl-2 inhibitor and a hypomethylating agent first, and then administering the FLT3 inhibitor sequentially; or
(iv) by administering a Bcl-2 inhibitor, or Bcl-2 inhibitor and hypomethylating agent separately from FLT3 inhibitor, regardless of the order.

In one particular example, a FLT3 inhibitor, and

Bcl-2 inhibitors or Bcl-2 inhibitors and hypomethylating agents administered in combination with FLT3 inhibitors all may be in a therapeutically effective amount, respectively.

In one specific example,
(a) co-formulation of FLT3 inhibitors and Bcl-2 inhibitors, or FLT3 inhibitors and Bcl-2 inhibitors and hypomethylating agents; or
(b) the FLT3 inhibitor, the Bcl-2 inhibitor, or the FLT3 inhibitor and the Bcl-2 inhibitor and the hypomethylating agent may be formulated in separate dosage forms.

In one specific example,

As to a Bcl-2 inhibitor or a Bcl-2 inhibitor and a hypomethylating agent administered in combination with a composition comprising the FLT3 inhibitor,
(a) at least one is administered orally, or
(b) at least one may be administered parenterally.

In addition, the FLT3 inhibitor may be administered orally or parenterally.

In one specific example,

the FLT3 inhibitor is included in a therapy effective amount, and

it may be administered in combination with a therapy effective amount of Bcl-2 inhibitor, or a Bcl-2 inhibitor and a hypomethylating agent, respectively.

Another aspect provides a pharmaceutical kit in which the pharmaceutical composition is administered simultaneously, sequentially, in reverse order, or separately. According to one specific example, the pharmaceutical composition may be provided as part of a kit. For example, the kit may improve patient compliance or improve accuracy or convenience in preparing a composition for administration according to one embodiment. The kit may further include additional components for administering the composition according to one embodiment to a subject, such as a pharmaceutically acceptable carrier (e.g., sterile diluent). The kit may include package inserts or other information useful for administration to a subject described herein (e.g., prescription information).

Each component in the kit may be supplied in a separate, individual container. Alternatively, or additionally, the components of the compositions described herein may be supplied in a single container. In such a case, the container may be a container prepared for administration to a patient in need thereof, for example, an ampoule or syringe.

The contents of the kit may be provided in sterile form. The kit and its contents may be provided in a form ready for administration to the subject in need thereof. In such instances, the components of the combination of the kit are supplied as a formulation and optionally in a user administration device such that administration requires little additional action by the user. In the case that the kit comprises an administration device, such devices may be known and understood by those skilled in the practice for the routes of administration described herein, such as, but not limited to, syringes, pumps, bags, cups, inhalers, droppers, patches, creams, or syringes.

### 4. Target Disease

One aspect of the present disclosure provides a method for treating acute myeloid leukemia using the pharmaceutical composition and/or combination described above.

In embodiments, the present disclosure provides a method for treating acute myeloid leukemia in a subject in need thereof, comprising administering to the subject a therapeutically effective amount of a compound of Chemical Formula 1, or a pharmaceutically acceptable salt thereof, solvate thereof, stereoisomer thereof, tautomer thereof, or combination thereof and (i) a B-cell lymphoma-2 (Bcl-2) inhibitor or (ii) Bcl-2 inhibitor and a hypomethylating agent (HMA):

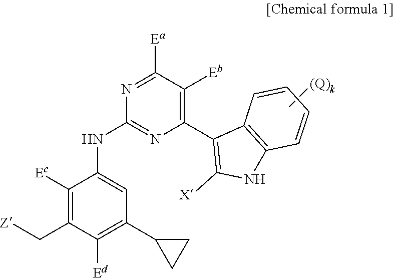

wherein in formula 1:

E^{a} is hydrogen, hydroxy or C1-4 alkoxy;

E^{b} is hydrogen, halogen, C1-4 alkyl or C1-4 fluoroalkyl;

E^{c} and E^{d} are hydrogen or hydroxy independently of each other;

X′ is hydrogen or hydroxy;

k is an integer from 1 to 2;

each Q is a hydroxy, halogen, C1-4 alkyl, hydroxy C1-4 alkyl or C1-4 alkoxy independently of each other;

Z′ is a monovalent functional group shown in Chemical Formula 2;

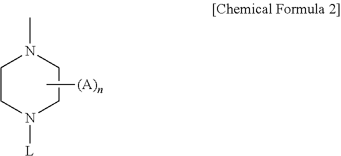

wherein, in Chemical Formula 2,

each A is a functional group independently selected from hydroxy, C1-4 alkyl, and hydroxy C1-4 alkyl, where at least one A is C1-4 alkyl;

n is an integer from 1 to 2; and

L is hydrogen, C1-4 alkyl, hydroxy or hydroxy C1-4 alkyl.

In embodiments, the present disclosure provides a method for treating acute myeloid leukemia in a subject in need thereof, comprising administering to the subject a therapeutically effective amount of a compound of Chemical Formula 3, or a pharmaceutically acceptable salt thereof, solvate thereof, stereoisomer thereof, tautomer thereof, or combination thereof and (i) a B-cell lymphoma-2 (Bcl-2) inhibitor or (ii) Bcl-2 inhibitor and a hypomethylating agent (HMA):

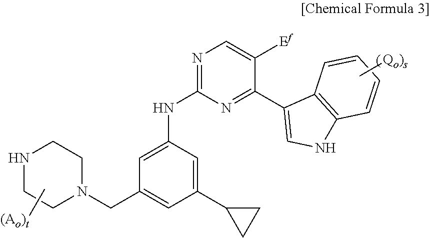

wherein:

E^{f} is fluorine, chlorine, bromine or iodine;

Q^{o} is hydroxy, halogen, C1-4 alkyl, hydroxy C1-4 alkyl or C1-4 alkoxy;

s is an integer from 1 to 2;

A^{o} is a functional group selected from hydroxy, C1-4 alkyl and hydroxy C1-4 alkyl; and

t is an integer from 1 to 2.

In embodiments, the present disclosure provides a method for treating acute myeloid leukemia in a subject in need thereof, comprising administering to the subject an effective amount of the compound:

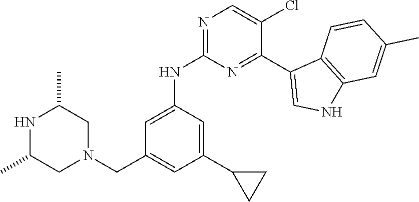

or a pharmaceutically acceptable salt thereof, solvate thereof, stereoisomer thereof, tautomer thereof, or combination thereof and (i) a B-cell lymphoma-2 (Bcl-2) inhibitor or (ii) Bcl-2 inhibitor and a hypomethylating agent (HMA).

In embodiments, the acute myeloid leukemia is relapsed or treatment-refractory (R/R) AML.

In embodiments, the acute myeloid leukemia is relapsed or treatment-refractory (R/R) AML in a subject who failed prior therapy with other FLT3 inhibitors.

In embodiments, the subject has one or more FLT3 mutations.

In embodiments, the subject has a TP53 mutation.

Following one example, the pharmaceutical composition has an excellent therapeutic effect on acute myeloid leukemia having an FMS-like tyrosine kinase 3 (FLT3) mutation, which leads to a high risk of recurrence after treatment, a poor prognosis, and a decrease in overall survival rate. Following the example, the pharmaceutical composition shows clinical benefits even in patients with acute myeloid leukemia who are resistant to conventional therapeutic agents.

In approximately 30% of AML patients, activated mutations in the internal tandem duplication (ITD) and tyrosine kinase domain (TKD) point mutations of FLT3 are reported as oncogenic driver mutations. For example, the mutation of the TKD may be one that further includes the internal tandem duplication.

In one specific example, the acute myeloid leukemia may be an acute myeloid leukemia having a FLT3 mutation.

In embodiments, the FLT3 mutation may be a FLT3 mutation in the internal tandem duplication (ITD) or one or more activating point mutations such as D835Y, D835V, 1836.

In one specific example, the acute myeloid leukemia may be a mutant FLT3 polynucleotide-positive acute myeloid leukemia, internal tandem duplication (ITD) positive acute myeloid leukemia in the FLT3 gene, or an acute myeloid leukemia having a FLT3 point mutation.

In one specific example, the acute myeloid leukemia may have a mutation in the tyrosine kinase domain (TKD) (FLT3-TKD) of the FLT3 amino acid sequence.

In one specific example, the FLT3-TKD mutation may further comprise an internal tandem duplication (ITD).

The mutation of FLT3-TKD may include one or more amino acid mutations in the positional region 823 to 861 of the FLT3 amino acid sequence. The mutation of the TKD may include a mutation of at least one amino acid selected from the group consisting of numbers 835, 836, and 842 of the FLT3 amino acid sequence. For example, the mutation of the TKD may include a mutation of amino acid 835 in the FLT3 amino acid sequence. For example, the mutation in TKD may be one in which the aspartic acid No. 835 of the FLT3 amino acid sequence is substituted with valine, tyrosine, histidine, glutamic acid, or asparagine. For example, the mutation of TKD may be one in which isoleucine 836 of the FLT3 amino acid sequence is substituted with leucine or aspartic acid. As another example, the mutation of TKD may be one in which tyrosine 842 of the FLT3 amino acid sequence is substituted with cysteine or histidine. Also, the mutation may be FLT3 (D835Y).

The FLT3-TKD mutation may have a mutation in at least one amino acid selected from the group consisting of 621, 627, 676, 691, and 697 of the FLT3 amino acid sequence. For example, the mutation of TKD may be one in which phenylalanine at position 691 of the FLT3 amino acid sequence is substituted with leucine. For example, the mutation may be FLT3 (F691L).

The mutation of the TKD may be one that further comprises an internal tandem duplication (ITD). For example, the mutation may be FLT3 (ITD/D835Y) or FLT3 (ITD/F691L).

In one specific example, the FLT3-TKD mutation may include any one selected from FLT3 (D835Y), FLT3 (F691L), FLT3 (F691L/D835Y), FLT3 (ITD/D835Y), FLT3 (ITD/F691L), and combinations thereof.

Following one specific example, as to 5-chloro-N-(3-cyclopropyl-5-(((3R,5S)-3,5-dimethylpiperazin-1-yl) methyl) phenyl)-4-(6-methyl-1H-indol-3-yl) pyrimidin-2-amine as a FLT3 inhibitor, the overcoming of tolerance and the therapeutic effect due to a FLT3 mutation are verified in an In vivo study using Ba/F3 cells expressed in FLT3 ITD/F691L or FLT3 ITD/D835Y xenograft mouse models.

Following one specific example, the FLT3 inhibitor shows an effect that can overcome the resistance of acute myeloid leukemia (AML) treatment. For example, the FLT3 inhibitor exhibits inhibitory activity against drug-resistant point mutants of FLT3 (D835Y, F691L, or F691L/D835Y) caused by the D835Y and F691L point mutations obtained in FLT3-TKD. In one particular example, the mutation of TKD may be one in which aspartic acid at position 835 of the FLT3 amino acid sequence is substituted with tyrosine. In another specific example, the mutation may be FLT3 (D835Y), or FLT3 (ITD/D835Y). In a specific example, the mutation of the TKD may be a leucine substitution for phenylalanine at position 691 of the FLT3 amino acid sequence. The mutation may be FLT3 (F691L) or FLT3 (ITD/F691L).

Following one specific example, as to FLT3 inhibitor, overcoming resistance and the therapeutic effect due to FLT3 mutation is verified through standard proliferation assay, immunoblotting, and apoptosis analysis, as an in vitro site-directed competition binding assay using an AML resistant cell line.

Following one specific example, the FLT3 inhibitor strongly inhibits FLT3 (ITD/D835Y) and FLT3 (ITD/F691L) mutations in preclinical evaluation. Following one specific example, the FLT3 inhibitor exhibits high in vitro binding affinity in both mutations, and exhibits strong inhibitory activity in in vitro and in vivo using Ba/F3 cell lines expressing FLT3 (ITD/D835Y) or FLT3 (ITD/F691L). Moreover, following one specific example the FLT3 inhibitor exhibits high cytotoxic efficacy in the MOLM-14 cell line harboring the FLT3 ITD, and can overcome the FLT3 ligand (FL)-related drug resistance mechanism.

Following another specific example, the FLT3 inhibitor can strongly inhibit phosphorylation of SYK, STAT3 and STAT5 in KG-la cells.

Additionally, following one specific example, the FLT3 inhibitor may be administered in combination with other one or more leukemia treatment drugs such as Bcl-2 inhibitors, or additional treatment drug(s) of Bcl-2 inhibitors and hypomethylating agents to give synergistic effects.

As used herein, the term “mutation” or “sequence variation” may refer to a substitution, insertion, deletion, duplication, or rearrangement.

As used herein, the term “mutation” refers to a substitution of a residue in a sequence, e.g., a nucleic acid or amino acid sequence, with another residue, or an insertion, deletion, duplication or rearrangement of one or more residues in a sequence. For example, the mutation includes a point mutation. In another example, the mutation includes a missense mutation or a nonsense mutation.

As used herein, the term “mutant” refers to the altered nucleic acid or polypeptide, or a cell or organism containing or expressing such a changed nucleic acid or polypeptide.

As used herein, the term “cancer” refers to a broad group of various diseases characterized by the uncontrolled growth of abnormal cells in the body. “cancer” or “cancer tissue” may include a tumor.

As used herein, the term “subject” encompasses mammals and non-mammals, including humans. Examples of mammals include, but are not limited to, humans, chimpanzees, apes, monkeys, cattle, horses, sheep, goats, pigs; rabbits, dogs, cats, rats, mice, guinea pigs, and the like. Examples of non-mammals include, but are not limited to, birds, fish, and the like.

The term “combination therapy” as used herein refers to the administration of two or more kinds of therapeutic agents for treating a therapeutic condition or disorder described herein. Such administration involves simultaneous administration of these therapeutic agents in a single capsule or tablet with a fixed ratio of active ingredients, or in multiple or individual containers for each active ingredient (e.g., capsules and/or intravenous formulations). Also, such administration includes the use of each type of therapeutic agent in a sequential manner, at approximately the same time or at different times. In either case, the treatment regimen provides the beneficial effect of the pharmaceutical combination treating the condition or disorder described herein.

As used herein, the terms “treating”, “to treat”, “treat” or “treatment” refer to limiting, delaying, arresting, reducing or reversing the progression or severity of an existing symptom, disease, condition, or disorder.

The term “to” used in the present specification refers to a range including values described as lower and upper limits before and after the term “to”. It refers to a section between the numerical values before and after the term “to” with those values included in the section. The numerical value may be a range in which any number of upper and/or lower limits are selected and combined.

The industrial applicability herein is shown by the positive impact in one or more of the studies described above, including the description of one or more parameters of the utility of this combination therapy.

Hereinafter, the present invention will be described in more detail with the following examples and experimental examples. However, these examples and experimental examples are only for helping the understanding of this invention, and the scope of this invention is not limited thereto in any sense.

### EXAMPLES

### Example 1. MV-4-11 Cell Line Xenografted Mouse Model

In a mouse model xenografted with the cell line MV-4-11 with FLT3 ITD/ITD isogenic mutation (ATCC, CRL-9591), a comparative or combination efficacy test of the FLT3 inhibitor, 5-chloro-N-(3-cyclopropyl-5-(((3R,5S)-3,5-dimethylpiperazin-1-yl) methyl) phenyl)-4-(6-methyl-1H-indol-3-yl) pyrimidin-2-amine (hereinafter ‘Compound A’), and the Bcl-2 inhibitor, 4-[4-[[2-(4-chlorophenyl)-4,4-dimethylcyclohexen-1-yl]methyl] piperazin-1-yl]-N-[3-nitro-4-(oxan-4-ylmethylamino) phenyl] sulfonyl-2-(1H-pyrrolo [2,3-b]pyridin-5-yloxy) benzamide (hereinafter ‘venetoclax’), was conducted.

MV-4-11 cell lines were purchased from the American Type Culture Collection (ATCC). To build a mouse model xenografted with this MV-4-11 cell line, a 5-week-old male BALB/c Nude mouse (hereinafter referred to as “Nude Mouse”) was purchased from Charles River Laboratories Japan, Inc.

The MV-4-11 cell line was injected subcutaneously in a nude mouse with 5×10\hat{ }6 cells/0.15 mL/mouse, and was allowed to grow. Mice with a tumor volume of 80 to 300 mm\hat{ }3 (length×width\hat{ }2×0.5) were selected on the day of administration, separated into 4 groups (7/group) and administered for a total of 21 days so that the average tumor volume in each group was almost the same.

The control group was administered DMSO/PEG400/DW (ratio=0.5/2/7.5, volume/volume/volume) mixed solution orally once a day, and the compound A group was administered 3 mg/kg/day once a day. It was orally administered, and the venetoclax group was orally administered once a day at a dose of 100 mg/kg/day. In the combined group, compound A was administered orally once a day at a dose of 3 mg/kg/day, and venetoclax was administered orally once a day at a dose of 100 mg/kg/day. Each group was administered individual medications for 21 days.

The maximum inhibition rate (%) and the change in weight loss (%) of mice were calculated, and the experimental results are shown in Table 1. The Inhibition rate (IR) was obtained from “(1−mean of individual relative tumor volume in the drug treatment group/average of individual relative tumor volume in the control group)×100%”, and the maximum inhibition rate was the largest inhibition rate during the observation period. The change in weight loss (%) was calculated from “(1−weight on the day of measurement/weight on the start day of administration)×100%”. The relative tumor volume was calculated from “the tumor volume/initial tumor volume×100% at the date of measurement”.

TABLE 1

| | | Maximum | | Change in |
|---|---|---|---|---|
| Sample | Dose (mg/kg) | inhibition (%) | Significance (Day 21) | weight loss (%) |
| Control group | 0 | — | Not applicable | — |
| Compound A | 3 | 54.0 | Not applicable | — |
| Venetoclax | 100 | 76.1 | Not applicable | — |
| Compound A + Venetoclax | 3 + 100 | 97.4 | P < 0.0001 (vs. compound A) | — |
| | | | P < 0.0001 (vs. venetoclax) | |

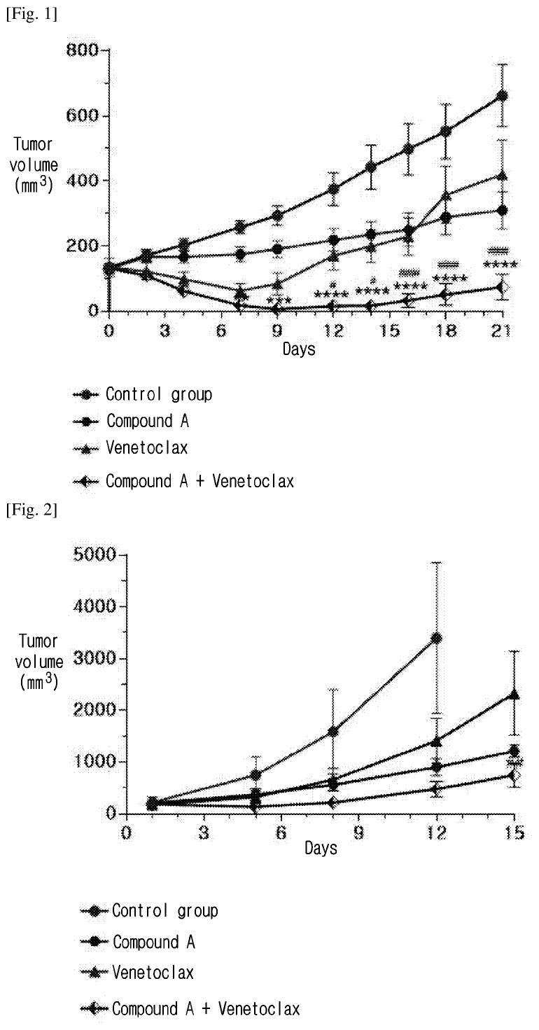

Additionally, the experimental results are shown in FIG. 1. This shows the tumor volume (mm\hat{ }3) measured after administration of each treatment solution or medication alone or in combination to nude mice xenografted with the MV-4-11 cell line. From the results of FIG. 1, the anti-tumor effect when the FLT3 inhibitor and the Bcl-2 inhibitor are co-administered can be seen. The Y-axis represents the mean tumor volume (mm\hat{ }3), and the X-axis represents the number of days of administration.

As shown in FIG. 1, the average tumor volume was measured for a total period of 21 days of drug administration in each treatment group, and an Inhibition Rate (IR) for evaluating anti-tumor effects was obtained from this. As a result, compared with the group administered only with compound A (compound A group) or the group administered only with venetoclax, the average tumor volume was significantly reduced in the combination group, and the tumor Inhibition Rate (IR) in the combination group increased. (maximum inhibition rate (MIR)=97.4% in the combination group, MIR=54.0% in the compound A group, MIR=76.1% in the venetoclax group).

As shown in FIG. 1, as a result of measuring and confirming the tumor volume according to drug administration, complete regression of the venetoclax group was observed in 3 out of 7 mice on the 9th day of administration, and complete regression of the combined group was observed in 6 out of 7 mice on the 9th day of administration. In the case of compound A group, complete regression was not observed, and partial regression (1 out of 7 mice) was observed on the 16th day of administration. Additionally, the results in FIG. 1 showed that the tumor volume in the combined group was significantly reduced compared to the single administration group of compound A or venetoclax, showing better anti-tumor efficacy in the combined administration group. (**p<0.01, ***p<0.001, and ****p<0.0001 vs. compound A alone; #p<0.05, and ####p<0.0001 vs. venetoclax alone; Sidk's test after two-way ANOVA comparison)

As such, from the experimental results using the mouse efficacy model in which the MV-4-11 cells shown in FIG. 1 and Table 1 were xenografted, compared to the group administered compound A, a FLT3 inhibitor, alone or the group administered venetoclax, a Bcl-2 inhibitor, alone, the reduction in the average tumor volume of the tumors with the FLT3 ITD/ITD homogeneous mutation significantly increased in the combination group of the FLT3 inhibitor and the Bcl-2 inhibitor, confirming superior and synergistic anti-tumor efficacy.

### Example 2. MOLM-13 Cell Line Xenografted Mouse Model

In a mouse model xenografted with the MOLM-13 (DSMZ no. ACC 554) cell line carrying the FLT3-WT/ITD heterogeneous mutation, a comparative or combination efficacy test of the FLT3 inhibitor, 5-chloro-N-(3-cyclopropyl-5-(((3R,5S)-3,5-dimethylpiperazine-1-yl) methyl) phenyl)-4-(6-methyl-1H-indol-3-yl) pyrimidin-2-amine (hereinafter ‘Compound A’) and the Bcl-2 inhibitor, venetoclax was conducted.

The MOLM-13 cell line was purchased from Deutsche Sammlung von Mikroorganismen und Zellkulturen (DSMZ) GmbH. To construct a mouse model xenografted with this MOLM-13 cell line, 5-week-old male nude mice were purchased from Charles River Laboratories Japan, Inc.

The MOLM-13 cell line was injected subcutaneously into nude mice at 5×10\hat{ }6 cells/0.2 mL/mouse, and was allowed to grow. Mice having a tumor volume of 55 to 415 mm\hat{ }3 (length×width\hat{ }2×0.5) were selected on the day of administration, and after having been divided into 4 groups (3 mice each in the control group and the compound A group, 4 mice each in the venetoclax group and the compound A and venetoclax combination group) so that the average tumor volume of each group was similar, the control group was administered for 11 days, and each drug administration group received individual medications for 14 days. The control group was measured for 12 days, and each drug administration group was measured for 15 days.

The control group received an oral administration of a mixed solution of DMSO/PEG400/DW (ratio=0.5/2/7.5, volume/volume/volume) once a day, the compound A group received an oral administration once a day at a dose of 10 mg/kg/day, and the venetoclax group received an oral administration once a day at a dose of 100 mg/kg/day. In the combined group, compound A was administered orally once a day at a dose of 10 mg/kg/day, and the venetoclax was administered orally once a day at 100 mg/kg/day.

The maximum inhibition rate (%) and the change in weight loss (%) of mice were calculated, and the experimental results are shown in Table 2. The inhibition rate (IR) was obtained from “(1−average of individual relative tumor volume in the drug treatment group/average of individual relative tumor volume in the control group)×100%”, and the maximum inhibition rate was the largest inhibition rate during the observation period. The change in weight loss (%) was calculated from “(1−body weight on the measurement day/weight on the start date of administration)×100%”. Relative tumor volume was calculated from “tumor volume on measurement day/initial tumor volume×100%”.

TABLE 2

| | | Maximum | | Change in |
|---|---|---|---|---|
| Sample | Dose (mg/kg) | inhibition (%) | Significance (Day 21) | weight loss (%) |
| Control group | 0 | — | Not applicable | — |
| Compound A | 10 | 68.1 | Not applicable | — |
| Venetoclax | 100 | 56.3 | Not applicable | — |
| Compound A + Venetoclax | 10 + 100 | 82.6 | P < 0.0001 (vs. venetoclax) | — |

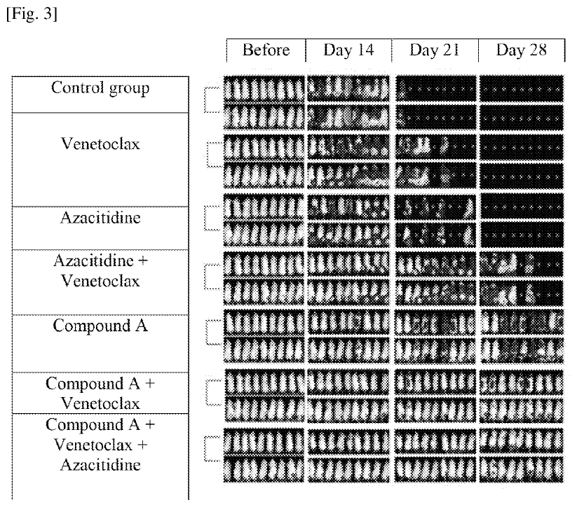

Additionally, the experiment results are shown in FIG. 2. It shows the tumor volume (mm\hat{ }3) measured after administration of each treatment solution or drug alone or in combination in nude mice xenografted with the MOLM-13 cell line. From the results shown in FIG. 2, the anti-tumor effect when the FLT3 inhibitor and the Bcl-2 inhibitor are co-administered can be seen. The Y-axis represents the average tumor volume (mm\hat{ }3), and the X-axis represents the number of days of administration.

As shown in FIG. 2, the average tumor volume was measured during the drug administration period in each treatment group, and the inhibition rate (IR) for evaluating the anti-tumor effect was obtained from this. As a result, compared with the group administered only with compound A or the group administered only with venetoclax, the mean tumor volume in the combination group was significantly reduced, and the inhibition rate (IR) in the combination group increased (maximum inhibition rate (MIR)=82.6% in the combination group, MIR=68.1% in the compound A group, MIR=56.3% in the venetoclax group).

As shown in FIG. 2, as a result of measuring and confirming the tumor volume according to drug administration, complete regression was not observed in the compound A and venetoclax groups, and complete regression in the combined group was observed in 1 out of 4 mice on the 7th day of administration. Additionally, in the results of FIG. 2, the tumor volume in the combined group was significantly reduced compared to the compound A group or the venetoclax group, showing better anti-tumor efficacy. (no significance vs. compound A alone; ##p<0.01 vs. venetoclax alone; Sidk's test performed after two-way ANOVA comparison)

As such, from the experimental results using the mouse efficacy model in which the MOLM-13 cells are xenografted as shown in FIG. 2 and Table 2, compared to the group administered only with compound A, an FLT3 inhibitor, or the group administered only with the Bcl-2 inhibitor, it was confirmed that, in the combination group of FLT3 inhibitor and Bcl-2 inhibitor, the decrease in average volume of tumors with FLT3 WT/ITD heterogeneity increased, showing better anti-tumor efficacy.

Additionally, from the results of Table 1 and Table 2 in Example 1, compared to the group administered only with compound A, an FLT3 inhibitor, or the group administered only with Bcl-2 inhibitor, it can be seen that the maximum inhibition rate (%) for tumors with FLT3-ITD mutations is increased in the combination group of FLT3 inhibitors and Bcl-2 inhibitors.

From the above results, an improved anti-tumor effect is shown for acute myeloid leukemia with FLT3-ITD mutation when the FLT3 inhibitor, 5-chloro-N-(3-cyclopropyl-5-(((3R,5S)-3,5-dimethylpiperazin-1-yl) methyl) phenyl)-4-(6-methyl-1H-indol-3-yl) pyrimidin-2-amine (hereinafter ‘Compound A’), and the Bcl-2 inhibitor, venetoclax, were used in combination.

### Example 3. MOLM-14 cell line orthotopically xenografted mouse model

In a mouse model in which MOLM-14 Luc/GFP (mother cell DSMZ no. ACC 777) cell line carrying the FLT3-WT/ITD heterogeneous mutation is orthotopically xenografted in the bone marrow, a comparative or combination efficacy test of the FLT3 inhibitor, 5-chloro-N-(3-cyclopropyl-5-(((3R,5S)-3,5-dimethylpiperazin-1-yl) methyl) phenyl)-4-(6-methyl-1H-indol-3-yl) pyrimidin-2-amine (hereinafter ‘Compound A’), and the Bcl-2 inhibitor, venetoclax, as well as the hypomethylating agent (HMA), 4-amino-1-[(2R,3R,4S,5R)-3,4-dihydroxy-5-(hydroxymethyl) oxolan-2-yl]-1,3,5-triazin-2-one (hereinafter ‘Azacitidine’) was conducted.

MOLM-14 cell line was purchased from Deutsche Sammlung von Mikroorganismen und Zellkulturen (DSMZ) GmbH, and CMV-Luciferase (firefly)-2A-GFP (Neo) transduced MOLM-14 Luc/GFP was prepared for luminescence measurement. In order to construct a mouse model in which the MOLM-14 Luc/GFP cell line was orthotopically xenografted into the bone marrow, 4-week-old male NOD/SCID mice were purchased from Central Institute for Experimental Animals (CIEA) in Japan.

The MOLM-14 Luc/GFP cell line was transplanted by intratibial injection of NOD/SCID mice at 2×10\hat{ }6 cells/0.03 mL/mouse and allowed to grow. In bioluminescence imaging, mice having an average total flux of 3.92×10\hat{ }8 (hereafter referred to as luminescence) were selected the day before administration, and after dividing into 6 groups (9 mice per group) so that the average luminescence of each group was similar, administration was carried out for 28 days, but if death occurred, administration was continued until the day before the death of each individual mouse.

The control group received oral administration of a mixed solution of DMSO/PEG400/DW (ratio=0.5/2/7.5, volume/volume/volume) once a day, and the compound A alone administration group received oral administration once a day at a dose of 15 mg/kg/day. The venetoclax alone administration group received oral administration once a day at a dose of 100 mg/kg/day. The azacitidine alone group was administered for 5 consecutive days from 0 to 4 days and every 3 weeks from 21 to 26 days at a dose of 3 mg/kg/day.

In the combination group of compound A and venetoclax, compound A was administered orally once a day at a dose of 15 mg/kg/day, and venetoclax was administered orally once a day at 100 mg/kg/day.

In the 3-drug combination group of compound A, venetoclax, and azicitidine, compound A was administered orally once a day at a dose of 15 mg/kg/day, venetoclax was administered orally once a day at a dose of 100 mg/kg/day, and azacitidine was administered through the caudal vein for 5 consecutive days from 0 to 4 days and every three weeks from 21 to 26 days at a dose of 3 mg/kg/day.

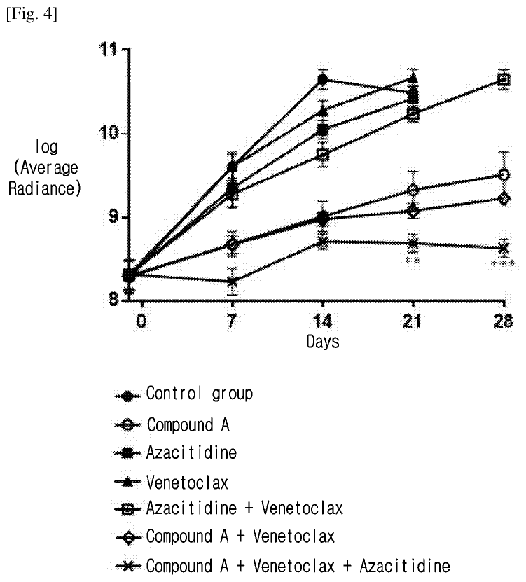

The experimental results are shown in FIGS. 3 and 4.

FIG. 3 shows a bioluminescence image measured after administering each treatment solution or drug alone or in combination in a NOD/SCID mouse in which the MOLM-14 Luc/GFP cell line is orthotopically xenografted into the bone marrow. In FIG. 3, the dead mouse is shown in a black background.

FIG. 4 shows the anti-tumor effect of administration of each treatment solution or drug alone or in combination in NOD/SCID mice in which the MOLM-14 Luc/GFP is orthotopically xenografted into the bone marrow.

The Y-axis of FIG. 4 represents the log average luminescence obtained from the luminescence measured in mice in each experimental group, and the X-axis represents the number of days of administration.

From the results of FIGS. 3 and 4, the anti-tumor effect when the FLT3 inhibitor, the Bcl-2 inhibitor and the hypomethylating agent are administered in combination can be seen.

As shown in FIG. 3, bioluminescence images were measured during the drug administration period in each treatment group, and quantified as in FIG. 4 from this in order to obtain a log average luminescence for evaluating the anti-tumor effect.

As a result, compared with the group administered with only compound A, the luminescence intensity decreased in the compound A and venetoclax 2 agent combination group and the compound A, venetoclax, and azacitidine 3 agent combination group. In the 3 agent combination group, there was a significant decrease in log average luminescence. (**p<0.01, ***p<0.001 vs. compound A alone; Dunnett's test performed after two-way ANOVA comparison)

As such, from the experimental results using the mouse efficacy model in which the MOLM-14 Luc/GFP cells were orthotopically xenografted into the bone marrow, shown in FIGS. 3 and 4, luminescence in tumors with FLT3 WT/ITD heterogeneous mutations was significantly decreased in the 3-agent combination group of FLT3 inhibitor, Bcl2 inhibitor and hypomethylating agent, compared with the groups administered with only the FLT3 inhibitor, compound A, with only the Bcl-2 inhibitor or hypomethylating agent and with FLT3 inhibitor and Bcl-2 inhibitor in combination, confirming that the 3-agent combination group showed better anti-tumor efficacy.

From the above results, use of the FLT3 inhibitor, 5-chloro-N-(3-cyclopropyl-5-(((3R,5S)-3,5-dimethylpiperazin-1-yl) methyl) phenyl)-4-(6-methyl-1H-indol-3-yl) pyrimidin 2-amine (hereinafter referred to as the ‘Compound A’), a Bcl-2 inhibitor, venetoclax, and a hypomethylating agent (HMA), Azacitidine, in combination showed an improved anti-tumor effect on acute myeloid leukemia having a FLT3-ITD mutation.

### Example 4. Compound A and Venetoclax Study

This study is an open-label, first-in-human, dose escalation, exploration, and expansion study of Compound A as a single agent and in combination with venetoclax in patients with relapsed or refractory AML. Cycle 1 in dose escalation is defined as 30 days, all other cycles lasting at least 28 days. Patients will receive oral Compound A at QD on a continuous basis, with the exception of Cycle 1 Day 2 in Part A. The study treatment can continue until a discontinuation criterion is met.

The initial dose level of Compound A as a single agent was 20 mg daily and the decision to dose escalate to the next dose level is made based on the assessment of safety variables including moderate toxicity (MT, a Grade 2 AE judged by the investigator to be related to study drug (except for hematologic toxicities)) or dose limiting toxicity (DLT). This study includes 3 parts: Part A: Dose Escalation; Part B: Dose Exploration; and Part C: Dose Expansion.

### Part A: Dose Escalation:

Part A includes the initial dose escalation cohorts. Provisional dose escalation scheme with the planned doses of Compound A are as follows: 20 mg, 30 mg, 40 mg, 60 mg, 80 mg, 120 mg, 160 mg, 200 mg, or 240 mg. Patients are treated daily in 28-day cycles except for the Cycle 1 (30 days). The DLT observation period is during Cycle 1 starting with the first dose on Day 1. Patients in Cycle 1 have PK sampling performed after receiving a single dose of the study drug on Day 1. This study follows an accelerated titration design. Dose levels are set at around 50% increments. One patient is treated at the starting dose level of 20 mg. If no DLT or MT is identified, the next patient is enrolled at double the dose level, i.e., dose level 3 (40 mg). This dose escalation approach will continue until the first instance of a DLT or MT (a Grade 2 AE judged by the investigator to be related to study drug (except for hematologic toxicities) occur.

After DLT evaluable patients are identified or MT is observed in each dose level in the accelerated titration design, data pertaining to dose escalation decisions are reviewed. Available data including demographics, AEs, laboratory assessments, dose administration, and any other pertinent information relevant to patient's safety will be reviewed. The determination will be made as to whether dose escalation should continue and, if so, at what dose level and schedule. This data review may be performed at any time. In addition, if safety assessment is required a data review may take place ad-hoc.

When a DLT or MT is observed in a patient, the dose escalation schedule will stop the double-dose level method and follow the next consecutive dose level in utilizing the modified 3+3 design. Modified 3+3 design testing each consecutive dose level may also be followed if recommended through the safety review meeting (RM) based on the review of available PK data.

After dose escalation design converting to 3+3 design, 3 patients are treated at each dose level. If no DLTs are observed among 3 patients, the subsequent patients are treated at the next dose level. If one DLT is observed among 3 patients in a dose level (1/3 DLTs observed), 3 more patients are enrolled at that dose level. If the 3 additional patients do not experience a DLT (5/6 DLTs observed), the next dose level is initiated. If 2 or more DLTs occur in a dose level (2/3 or 2/6 DLTs observed), the dose will ne considered intolerable and dose escalation will be stopped. MTD will be determined at the next lower level or, if appropriate, a dose between the highest tolerable one and the intolerable one will be further explored to determine MTD.

### Part B: Dose Exploration:

Part B is the dose exploration cohort. Patients will be treated daily in 28-day cycles. The DLT observation period is during Cycle 1 starting with the first dose on Day 1. At any dose level, if no DLTs are observed in the initial 3 patients (0/3 DLTs observed) in dose escalation cohort (Part A), the dose level will be expanded to enroll up to 6 patients (including initial 3 patients) and DLT assessment will be performed. If one or fewer DLTs are observed in the 6 patients (≤1/6 DLTs observed), the dose level continues to enroll patients up to 20 patients. If 2 or more DLTs occur among 6 patients (2/6 DLTs observed) in a dose level, the additional enrollment will be stopped. If one DLT is observed in the 6 patients (1/6 DLT observed) in the dose escalation cohort (Part A), up to 20 patients can be enrolled in the dose exploration cohort (Part B) at the dose level. The planned doses of Compound A are for Part B are also as follows: 20 mg, 30 mg, 40 mg, 60 mg, 80 mg, 120 mg, 160 mg, 200 mg, or 240 mg.

If both Part A and Part B are open concurrently, newly enrolled patients are assigned to Part A first. If multiple dose levels are simultaneously explored, patient enrollment is carried out preferentially at the lowest dose of the explored dose levels. As several complete responses are observed at 80 mg dose, the 40 mg dose level may be expanded up to a total of 20 patients to further explore the safety, PK, and activity at this dose level. Part B can occur concurrently with dose expansion in Part C.

At least half of the patients in Part B dose level have AML with FLT3 mutation (e.g., ITD or activating point mutations such as D835Y, D835V, 1836) including patients in Part A. If 10 FLT3-unmutated patients are enrolled at a dose level, that level will be closed to further enrollment of FLT3-unmutated patients. Patients with or without a documented FLT3 mutation will be enrolled and samples will be collected at screening visit to confirm or evaluate FLT3 mutation status. If the FLT3 mutation status at the time of enrollment is unknown, patients will be considered to have the FLT3 mutation status determined by their most recent prior genetic test. FLT3-ITD or TKD mutation will be determined with FDA-approved test or validated assay in central laboratory.

The safety in the dose exploration cohort (Part B) will be monitored using Bayesian logistic regression modeling, based on the DLT rate observed in patients from both Part A and Part B.

If at least one patient in Part A at any dose level achieves clinical response (CR, CR with partial hematological recovery (CRh), CR with incomplete platelet recovery (CRp), CR with incomplete hematological recovery (CRi), or partial remission (PR)), then the dose level can continue to enroll a minimum of 3 patients in either Part A or Part B. In parallel with the accelerated dose escalation, DLT assessment will be performed for the initial 3 patients at the dose level of exploration part for safety monitoring in the same scheme as in 3+3 design.

If no DLTs are observed in the initial 3 patients or the 3 following patients do not experience a DLT after one patient in the initial 3 patients experiences a DLT (0/3 or 1/6 DLTs observed), the dose level will continue to enroll additional patients. Additionally, if fewer than 2 responses (composite CR [CR+CRh+CRi+CRp] (CRc)+PR) are achieved in 12 patients who complete 2 treatment cycles, the dose level will stop further enrollment. Otherwise, the dose level continues to enroll patients up to 20 evaluable patients. For the patients in Part B, Bayesian logistic regression model will be also applied as supportive analysis for safety assessment.

### Part C: Dose Expansion:

Part C is the dose expansion cohort to determine safety and tolerability of Compound A plus venetoclax. Patients are assigned to either the Compound A single agent treatment group or the Compound A plus venetoclax combination treatment group. Within each group, approximately half of patients have FLT3 mutations, and the other half are FLT3-unmutated. Among the FLT3-mutated patients in the Compound A single agent treatment group, at least 16 (evaluable) patients have received prior treatment with a FLT3 inhibitor. Of the FLT3-unmutated patients in the Compound A single agent treatment group, at least 12 (evaluable) patients have a TP53 mutation or complex karyotype. The initial single agent dose of Compound A is 120 mg daily, and the starting dose in the Compound A plus venetoclax treatment group is 80 mg daily. Patients are assigned to a treatment group based on the number of slots available. Treatment will be in 28-day cycles, and patients receive daily dosing with Compound A in all treatment groups. Patients not evaluable for response assessment may be replaced. For the single agent group, after 6 patients have completed Cycle 1, an SRM will be held to complete a safety review that includes all subjects in the study to date. Recommendations of dosage adjustments based on the availability of safety information may be made at the SRM.

Response assessments will be performed on Cycle 1 Day 15 and at subsequent points depending on response. Events that occur in Part C that would be considered a DLT if occurred in Part A or Part B will be reviewed and assessed at the SRM. For the combination treatment group, a safety review meeting will be held after each set of 3 patients complete Cycle 1. The SRM can recommend changes to the dosing schedule for subsequent patients.

In both Part A and Part B, a patient that receives less than 80% of the intended dose during Cycle 1 (e.g., misses 6 daily doses or leaves the study for reasons other than a DLT) will not be evaluable for DLT and will be replaced by another patient in the dose level. In addition, if after enrollment any patient is found not to fulfill any inclusion/exclusion criteria that would adversely affect safety or efficacy evaluation of that patient, they may be replaced after discussion between the Principal Investigator and Medical Monitor. Based on evaluation of the primary objective, additional cohorts with potentially modified target patients and regimen of study drug may be accrued.

For Part C, patients may be replaced if they are deemed not evaluable for response by Cycle 3 Day 1 (have not received 2 cycles of treatment or have discontinued for a reason other than disease progression).

Provisional dose escalation scheme with the planned doses for Part A is presented. However, this plan may change based on the recommendations from the Cohort Review Meeting.

DLT assessment will be determined. A DLT is defined as any of the following events that occur within Cycle 1 starting with the first dose taken on Day 1 for both Part A and Part B, and that is considered to be related to study drug.

Any Grade ≥on-hematologic r extramedullary oxicity.

The following exceptions are noted:
a. Alopecia, anorexia, or fatigue of any grade.
b. Grade 3 nausea or vomiting or diarrhea if not requiring hospitalization, TPN, or tube feeding.
c. Grade 3 fever with neutropenia, with or without infection.
d. Grade 3 infection.
e. Hematologic toxicity will not be considered as a DLT.

However, prolonged myelosuppression defined as ANC<500 for more than 21 days off therapy in the absence of evidence of active leukemia in the marrow or blood will be considered as a DLT.

Any Grade 4 organ toxicity is a DLT.

Monitoring for excess toxicity is to continue to be performed in Part C. All available safety, tolerability, and PK data is reviewed for patients in the combination treatment group to determine if any adjustments need to be made to the venetoclax and/or Compound A doses for subsequent patients.

The study will indicate that the venetoclax and Compound A is both safe and tolerable to a patient.

So far, the present invention has been focused on specific examples thereof. Those of ordinary skill in the practice to which this invention pertains will appreciate that this invention may be implemented in a modified form without departing from its essential characteristics. Therefore, the disclosed examples are to be considered in an illustrative rather than a restrictive sense. The scope of the present invention is indicated in the claims rather than the foregoing description, and all differences within the scope equivalent thereto should be construed as being included in the present invention.

### EMBODIMENTS

[Embodiment 1] A pharmaceutical composition containing an Fms-like tyrosine kinase-3 (FLT3) inhibitor, the FLT3 inhibitor is a compound selected from the following compounds of Chemical Formula 1, their stereoisomers, their tautomers, and combinations thereof, and the composition has the characteristic of being administered in combination with a B-cell lymphoma-2 (Bcl-2) inhibitor or with a Bcl-2 inhibitor and hypomethylating agent (HMA) for the treatment of acute myeloid leukemia (AML) treatment.

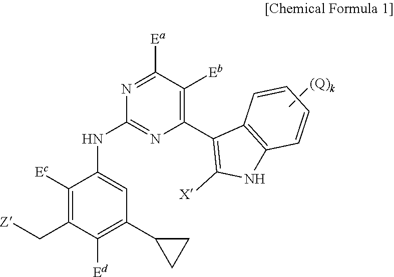

In Chemical Formula 1,

Ea is hydrogen, hydroxy or C1-4 alkoxy;

Eb is hydrogen, halogen, C1-4 alkyl or C1-4 fluoroalkyl;

Ec and Ed are hydrogen or hydroxy, independently of each other;

X′ is hydrogen or hydroxy;

k is an integer from 1 to 2;

Each Q is hydroxy, halogen, C1-4 alkyl, hydroxy C1-4 alkyl or C1-4 alkoxy, independently of each other;

Z′ is a monovalent functional group shown in Chemical Formula 2;

k is an integer from 1 to 2;

Each Q is a hydroxy, halogen, C1-4 alkyl, hydroxy C1-4 alkyl or C1-4 alkoxy independently of each other;

Z′ is a monovalent functional group represented by Chemical Formula 2;

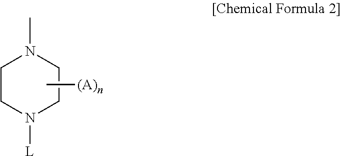

In this case, in Chemical Formula 2,

each A is a functional group selected from hydroxy, C1-4 alkyl and hydroxy C1-4 alkyl, independently of each other, wherein at least one A is C1-4 alkyl;

n is an integer from 1 to 2;

L is hydrogen, C1-4 alkyl, hydroxy or hydroxy C1-4 alkyl.

[Embodiment 2] The pharmaceutical composition according to embodiment 1, wherein the FLT3 inhibitor is a compound selected from the compound of Chemical Formula 3, its stereoisomers, tautomers, and combinations thereof.

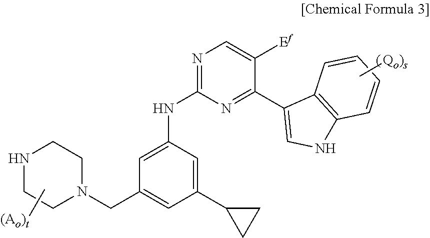

In Chemical Formula 3,

Ef is fluorine, chlorine, bromine or iodine;

Qo is hydroxy, halogen, C1-4 alkyl, hydroxy C1-4 alkyl or C1-4 alkoxy;

s is an integer from 1 to 2;

Ao is a functional group selected from hydroxy, C1-4 alkyl and hydroxy C1-4 alkyl;

t is an integer from 1 to 2.

[Embodiment 3] The pharmaceutical composition according to embodiment 1, wherein the FLT3 inhibitor is 5-chloro-N-(3-cyclopropyl-5-(((3R,5S)-3,5-dimethylpiperazine-1-yl) methyl) phenyl)-4-(6-methyl-1H-indol-3-yl) pyrimidin-2-amine

[Embodiment 4] The pharmaceutical composition according to embodiment 1, wherein the Bcl-2 inhibitor is any one selected from venetoclax, navitoclax, obatoclax, and combinations thereof.

[Embodiment 5] The pharmaceutical composition according to embodiment 1, wherein the hypomethylating agent (HMA) is any one selected from azacitidine, decitabine, idarubicin, and combinations thereof.

[Embodiment 6] The pharmaceutical composition according to embodiment 1, comprising the compound of Chemical Formula 1 as an FLT3 inhibitor and administered in combination with venetoclax

[Embodiment 7] The pharmaceutical composition according to embodiment 2, comprising the compound of Chemical Formula 3 as an FLT3 inhibitor and administered in combination with venetoclax

[Embodiment 8] The pharmaceutical composition according to embodiment 3, containing 5-chloro-N-(3-cyclopropyl-5-((3R,5S)-3,5-dimethylpiperazine-1-yl) methyl) phenyl)-4-(6-methyl-1H-indol-3-yl) pyrimidine-2-amine as an FLT3 inhibitor and administered in combination with venetoclax

[Embodiment 9] The pharmaceutical composition according to embodiment 1, including the compound of Chemical Formula 1 as an FLT3 inhibitor and co-administered with at least one hypomethylating agent selected from azacitidine, desitabin, and idarubicin, and venetoclax

[Embodiment 10] The pharmaceutical composition according to embodiment 2, including the compound of Chemical Formula 3 as an FLT3 inhibitor and co-administered with at least one hypomethylating agent selected from azacitidine, desitabin, and idarubicin, and venetoclax

[Embodiment 11] The pharmaceutical composition according to embodiment 3, containing 5-chloro-N-(3-cyclopropyl-5-(((3R,5S)-3,5-dimethylpiperazine-1-yl) methyl) phenyl)-4-(6-methyl-1H-indol-3-yl) pyrimidin-2-amine as an FLT3 inhibitor and co-administered with at least one hypomethylating agent selected from azacitidine, desitabin, and idarubicin, and venetoclax

[Embodiment 12] The pharmaceutical composition according to embodiment 3, containing 5-chloro-N-(3-cyclopropyl-5-(((3R,5S)-3,5-dimethylpiperazine-1-yl) methyl) phenyl)-4-(6-methyl-1H-indol-3-yl) pyrimidin-2-amine as an FLT3 inhibitor and administered in combination with venetoclax and azacitidine

[Embodiment 13] The pharmaceutical composition according to embodiment 1, wherein the Bcl-2 inhibitor, or the Bcl-2 inhibitor and the hypomethylating agent co-administered with the composition comprising the FLT3 inhibitor are respectively administered
(i) concurrently with a FLT3 inhibitor;
(ii) sequentially after the FLT3 inhibitor is administered first;
(iii) in a form in which Bcl-2 inhibitor, or Bcl-2 inhibitor and hypomethylating agent are administered first, then FLT3 inhibitor sequentially; or
(iv) in a form in which the Bcl-2 inhibitor or the Bcl-2 inhibitor and the hypomethylating agent are administered, separately from the FLT3 inhibitor, in any order

[Embodiment 14] According to embodiment 1,
(a) Co-formulation of a FLT3 inhibitor and a Bcl-2 inhibitor, or a FLT3 inhibitor and a Bcl-2 inhibitor and a hypomethylating agent; or
(b) A pharmaceutical composition in which the FLT3 inhibitor and the Bcl-2 inhibitor, or the FLT3 inhibitor and the Bcl-2 inhibitor and the hypomethylating agent are formulated in separate dosage form

[Embodiment 15] The pharmaceutical composition according to embodiment 1, wherein the Bcl-2 inhibitor, or the Bcl-2 inhibitor and the hypomethylating agent co-administered with the composition comprising the FLT3 inhibitor, are administered, respectively,
(a) in a form in which at least one is administered orally, or
(b) in a form in which at least one is administered parenterally

[Embodiment 16] The pharmaceutical composition according to embodiment 1, wherein the FLT3 inhibitor is included in a therapeutically effective amount, and is administered in combination with a therapeutically effective amount of Bcl-2 inhibitor, or a Bcl-2 inhibitor and a hypomethylating agent, respectively

[Embodiment 17] The pharmaceutical composition according to embodiment 1 or 11, wherein the acute myeloid leukemia is an acute myeloid leukemia having a FLT3 mutation

[Embodiment 18] The pharmaceutical composition according to embodiment 1 or 11, wherein the acute myeloid leukemia is a mutant FLT3 polynucleotide-positive acute myeloid leukemia, an internal tandem duplication (ITD) in the FLT3 gene-positive acute myeloid leukemia, or an acute myeloid leukemia having a FLT3 point mutation.

[Embodiment 19] The pharmaceutical composition according to embodiment 1 or 11, wherein the acute myeloid leukemia has a tyrosine kinase domain (TKD) (FLT3-TKD) mutation of the FLT3 amino acid sequence.

[Embodiment 20] The pharmaceutical composition according to embodiment 19, wherein the FLT3-TKD mutation further comprises internal tandem duplication (ITD).

[Embodiment 21] The pharmaceutical composition according to embodiment 19, wherein the FLT3-TKD mutation includes any one selected from FLT3 (D835Y), FLT3 (F691L), FLT3 (F691L/D835Y), FLT3 (ITD/D835Y), FLT3 (ITD/F691L), and combinations thereof.

[Embodiment 22] The pharmaceutical composition according to embodiment 1, wherein the FLT3 inhibitor is any one selected from the group consisting of the following compounds:
1) 5-chloro-N-(3-cyclopropyl-5-(((3R,5S)-3,5-dimethylpiperazin-1-yl) methyl) phenyl)-4-(6-fluoro-1H-indol-3-yl) pyrimidin-2-amine
2) 5-chloro-4-(6-chloro-1H-indol-3-yl)-N-(3-cyclopropyl-5-(((3R,5S)-3,5-dimethylpiperazine-1-yl) methyl) phenyl) pyrimidin-2-amine
3) 2-((2R,6S)-4-(3-((5-chloro-4-(6-fluoro-1H-indol-3-yl) pyrimidine-2-yl) amino)-5-cyclopropylbenzyl)-2,6-dimethylpiperazine-1-yl) ethan-1-ol
4) 2-((2R,6S)-4-(3-((5-chloro-4-(1H-indol-3-yl) pyrimidine-2-yl) amino)-5-[440] cyclopropylbenzyl)-2,6-dimethylpiperazine-1-yl) ethan-1-ol
5) 2-((2R,6S)-4-(3-((5-chloro-4-(6-methyl-1H-indol-3-yl) pyrimidin-2-yl) amino)-5-cyclo propylbenzyl)-2,6-dimethylpiperazin-1-yl) ethan-1-ol
6) (R)-5-chloro-N-(3-cyclopropyl-5-((3-methylpiperazin-1-yl) methyl) phenyl)-4-(1H-indol-3-yl) pyrimidine-2-amine
7) (R)-5-chloro-N-(3-cyclopropyl-5-((3-methylpiperazin-1-yl) methyl) phenyl)-4-(6-methyl-1H-indol-3-yl) pyrimidin-2-amine
8) 5-chloro-N-(3-cyclopropyl-5-(((3R,5S)-3,5-dimethylpiperazin-1-yl) methyl) phenyl)-4-(6-methyl-1H-indol-3-yl) pyrimidin-2-amine
9) 5-chloro-N-(3-cyclopropyl-5-(((3S,5R)-3-ethyl-5-methylpiperazine-1-yl) methyl) phenyl)-4-(6-methyl-1H-indol-3-yl) pyrimidin-2-amine
10) 5-chloro-N-(3-cyclopropyl-5-((3,5-dimethylpiperazin-1-yl) methyl) phenyl)-4-(6-methyl-1H-indol-3-yl) pyrimidin-2-amine
11) N-(3-cyclopropyl-5-(((3R,5S)-3,5-dimethylpiperazin-1-yl) methyl) phenyl)-4-(6-methyl-1H-indol-3-yl) pyrimidin-2-amine
12) N-(3-cyclopropyl-5-(((3R,5S)-3,5-dimethylpiperazin-1-yl) methyl) phenyl)-5-fluoro-4-(6-methyl-1H-indol-3-yl) pyrimidin-2-amine
13) N-(3-cyclopropyl-5-(((3R,5S)-3,5-dimethylpiperazin-1-yl) methyl) phenyl)-4-(1H-indol-3-yl)-5-methyl pyrimidin-2-amine
14) N-(3-cyclopropyl-5-(((3R,5S)-3,5-dimethylpiperazin-1-yl) methyl) phenyl)-5-methyl-4-(6-methyl-1H-indol-3-yl) pyrimidin-2-amine
15) N-(3-cyclopropyl-5-(((3R,5S)-3,5-dimethylpiperazin-1-yl) methyl) phenyl)-4-(6-methyl-1H-indol-3-yl)-5-(trifluoromethyl) pyrimidin-2-amine
16) 3-(5-chloro-2-((3-cyclopropyl-5-(((3R,5S)-3,5-dimethylpiperazine-1-yl) methyl) phenyl) amino) pyrimidin-4-yl)-1H-indol-6-yl) methanol
17) 5-chloro-N-(3-cyclopropyl-5-(((3R,5S)-3,5-dimethylpiperazin-1-yl) methyl) phenyl)-4-(5-methoxy-6-methyl-1H-indol-3-yl) pyrimidin-2-amine
18) 3-(5-chloro-2-((3-cyclopropyl-5-(((3R,5S)-3,5-dimethylpiperazin-1-yl) methyl) phenyl) amino) pyrimidin-4-yl)-6-methyl-1H-indol-5-ol
19) 3-(5-chloro-2-((3-cyclopropyl-5-(((3R,5S)-3,5-dimethylpiperazin-1-yl) methyl) phenyl) amino) pyrimidin-4-yl)-6-methylindolin-2-one
20) 5-chloro-N-(3-cyclopropyl-5-(((3R,5S)-3,5-dimethylpiperazin-1-yl) methyl) phenyl)-4-methoxy-6-(6-methyl-1H-indol-3-yl) pyrimidin-2-amine
21) 5-chloro-2-((3-cyclopropyl-5-(((3R,5S)-3,5-dimethylpiperazin-1-yl) methyl) phenyl) amino)-6-(6-methyl-1H-indol-3-yl) pyrimidin-4-ol
22) 3-(5-chloro-2-((3-cyclopropyl-5-(((3R,5S)-3,5-dimethylpiperazin-1-yl) methyl) phenyl) amino) pyrimidin-4-yl)-6-methyl-1H-indol-7-ol
23) 2-((5-chloro-4-(6-methyl-1H-indol-3-yl) pyrimidin-2-yl) amino)-4-cyclopropyl-6-(((3R,5S)-3,5-dimethylpiperazin-1-yl) methyl) phenol
24) 4-((5-chloro-4-(6-methyl-1H-indol-3-yl) pyrimidin-2-yl) amino)-2-cyclopropy-6-(((3R,5S)-3,5-dimethylpiperazin-1-yl) methyl) phenol
25) (R)-5-chloro-N-(3-cyclopropyl-5-((3,3,5-trimethylpiperazin-1-yl) methyl) phenyl)-4-(6-methyl-1H-indol-3-yl) pyrimidin-2-amine
26) ((2R,6R)-4-(3-((5-chloro-4-(6-methyl-1H-indol-3-yl) pyrimidin-2-yl) amino)-5-cyclopropylbenzyl)-6-methylpiperazin-2-yl) methanol
27) (R)-5-chloro-N-(3-cyclopropyl-5-((5-methyl-4,7-diazaspiro [2.5] oxtan-7-yl) methyl) phenyl)-4-(6-methyl-1H-indol-3-yl) pyrimidin-2-amine
28) 5-chloro-N-(3-cyclopropyl-5-(((3R,5R)-3,5-dimethylpiperazin-1-yl) methyl) phenyl)-4-(6-methyl-1H-indol-3-yl) pyrimidin-2-amine
29) 5-chloro-N-(3-cyclopropyl-5-(((3S,5S)-3,5-dimethylpiperazin-1-yl) methyl) phenyl)-4-(6-methyl-1H-indol-3-yl) pyrimidin-2-amine
30) 5-chloro-N-(3-cyclopropyl-5-(((3R,5S)-3,4,5-trimethylpiperazin-1-yl) methyl) phenyl)-4-(6-methyl-1H-indol-3-yl) pyrimidin-2-amine
31) (2R,6S)-4-(3-((5-chloro-4-(6-methyl-1H-indol-3-yl) pyrimidin-2-yl) amino)-5-cyclopropylbenzyl)-2,6-dimethylpiperazin-1-ol
32) (2R,6S)-4-(3-cyclopropyl-5-((4-(6-methyl-1H-indol-3-yl) pyrimidine-2-yl) amino) benzyl)-2,6-dimethylpiperazin-1-ol.

[Embodiment 23] A pharmaceutical composition comprising a Bcl-2 inhibitor, wherein the composition is administered in combination with a compound selected from 5-chloro-N-(3-cyclopropyl-5-(((3R,5S)-3,5-dimethylpiperazin-1-yl) methyl) phenyl)-4-(6-methyl-1H-indol-3-yl) pyrimidin-2-amine, its stereoisomers, tautomers, and combinations thereof; or with a compound selected from 5-chloro-N-(3-cyclopropyl-5-(((3R,5S)-3,5-dimethylpiperazin-1-yl) methyl) phenyl)-4-(6-methyl-1H-indol-3-yl0 pyrimidin-2-amine, its stereoisomers, tautomers, and combinations thereof and a hypomethylating agent for the treatment of acute myeloid leukemia.

1. A method of treating acute myeloid leukemia (AML) in a subject in need thereof, the method comprising administering to the subject a therapeutically effective amount of a compound of Chemical Formula 1, or a pharmaceutically acceptable salt thereof, solvate thereof, stereoisomer thereof, tautomer thereof, or combination thereof and (i) a B-cell lymphoma-2 (Bcl-2) inhibitor or (ii) Bcl-2 inhibitor and a hypomethylating agent (HMA); 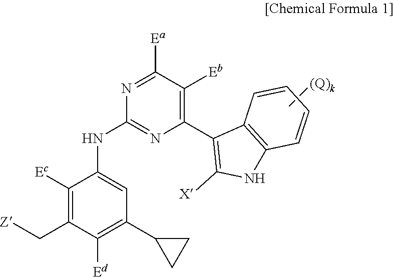
wherein in Chemical Formula 1,
Ea is hydrogen, hydroxy or C1-4 alkoxy;
Eb is hydrogen, halogen, C1-4 alkyl or C1-4 fluoroalkyl;
Ec and Ed are each independently hydrogen or hydroxy;
X′ is hydrogen or hydroxy;
k is an integer from 1 to 2;
each Q is independently of each other hydroxy, halogen, C1-4 alkyl,
hydroxy C1-4 alkyl or C1-4 alkoxy;
Z′ is a monovalent functional group represented by Chemical Formula 2; 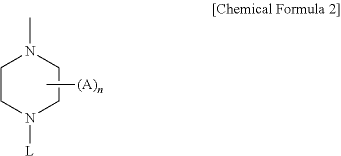
wherein in Chemical Formula 2,
each A is a functional group selected from hydroxy, C1-4 alkyl and hydroxy C1-4 alkyl, independently of each other, wherein at least one A is C1-4 alkyl;
n is an integer from 1 to 2; and
L is hydrogen, C1-4 alkyl, hydroxy or hydroxy C1-4 alkyl.

2. The method of claim 1, wherein the compound of Chemical Formula 1 is a Compound of Formula 3, or a pharmaceutically acceptable salt thereof, solvate thereof, stereoisomer thereof, tautomer thereof, or combination thereof;
thereof; 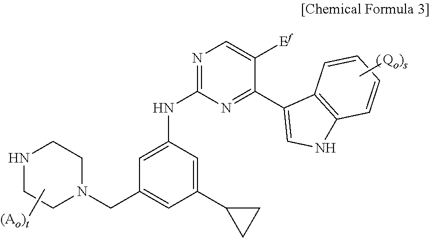
wherein in Chemical Formula 3,
Ef is fluorine, chlorine, bromine or iodine;
Qo is hydroxy, halogen, C1-4 alkyl, hydroxy C1-4 alkyl or C1-4 alkoxy;
s is an integer from 1 to 2;
Ao is a functional group selected from hydroxy, C1-4 alkyl and hydroxy C1-4 alkyl; and
t is an integer from 1 to 2.

3. The method of claim 1, wherein the compound of Chemical Formula 1 is the compound: 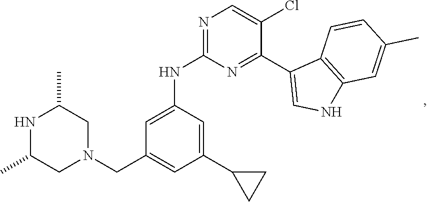
or a pharmaceutically acceptable salt thereof, solvate thereof, stereoisomer thereof, tautomer thereof, or combination thereof.

4. The method of claim 1, wherein the Bcl-2 inhibitor is selected from the group consisting of venetoclax, navitoclax, obatoclax, and combinations thereof.

5. The method of claim 1, wherein the hypomethylating agent (HMA) is selected from the group consisting of azacitidine, decitabine, idarubicin, and combinations thereof

6. The method of claim 1, wherein the compound of Chemical Formula 1 is administered in combination with a Bcl-2 inhibitor.

7. The method of claim 1, wherein the compound of Chemical Formula 1 is administered in combination with a Bcl-2 inhibitor and a hypomethylating agent (HMA).

8. The method of claim 1, wherein the compound of Chemical Formula 1 is administered in combination with venetoclax.

9. The method of claim 2, wherein the compound of Chemical Formula 3 is administered in combination with venetoclax.

10. The method of claim 3, wherein the compound is administered in combination with venetoclax.

11. The method of claim 1, wherein the compound of Chemical Formula 1 is administered in combination with venetoclax and at least one hypomethylating agent selected from azacitidine, decitabine, and idarubicin.

12. The method of claim 2, wherein the compound of Chemical Formula 3 is administered in combination with venetoclax and at least one hypomethylating agent selected from azacitidine, decitabine, and idarubicin.

13. The method of claim 3, wherein the compound is administered in combination with venetoclax and at least one hypomethylating agent selected from azacitidine, decitabine, and idarubicin.

14. The method of claim 3, wherein the compound is administered in combination with venetoclax and azacitidine.

15. The method of claim 1, wherein the (i) the Bcl-2 inhibitor, or (ii) the Bcl-2 inhibitor and the hypomethylating agent is:
(i) administered concurrently with the compound of Chemical Formula 1;
(ii) administered sequentially after the compound of Chemical Formula 1 is administered first;
(iii) administered first, before sequential administration of the compound of Chemical Formula 1; or
(iv) in a form in which the Bcl-2 inhibitor or the Bcl-2 inhibitor and the hypomethylating agent are administered, separately from the compound of Chemical Formula 1, in any order.

16. The method of claim 1, wherein the compound of Chemical Formula 1 and the Bcl-2 inhibitor or Bcl-2 inhibitor and a hypomethylating agent are co-formulated.

17. The method of claim 1, wherein the compound of Chemical Formula 1 and the Bcl-2 inhibitor or Bcl-2 inhibitor and a hypomethylating agent are administered in separate dosage forms.

18. The method of claim 1, wherein the Bcl-2 inhibitor, or the Bcl-2 inhibitor and the hypomethylating agent and the compound of Chemical Formula 1 are administered, respectively,
(a) in a form in which at least one is administered orally, or
(b) in a form in which at least one is administered parenterally.

19. The method of claim 1, wherein the compound of Chemical Formula 1 is selected from the group consisting of:
1) 5-chloro-N-(3-cyclopropyl-5-(((3R,5S)-3,5-dimethylpiperazin-1-yl) methyl) phenyl)-4-(6-fluoro-1H-indol-3-yl) pyrimidin-2-amine;
2) 5-chloro-4-(6-chloro-1H-indol-3-yl)-N-(3-cyclopropyl-5-(((3R,5S)-3,
5-dimethylpiperazine-1-yl) methyl) phenyl) pyrimidin-2-amine;
3) 2-((2R,6S)-4-(3-((5-chloro-4-(6-fluoro-1H-indol-3-yl) pyrimidine-2-yl) amino)-5-cyclopropylbenzyl)-2,6-dimethylpiperazine-1-yl) ethan-1-ol;
4) 2-((2R,6S)-4-(3-((5-chloro-4-(1H-indol-3-yl) pyrimidine-2-yl) amino)-5-cyclopropylbenzyl)-2,6-dimethylpiperazine-1-yl) ethan-1-ol;
5) 2-((2R,6S)-4-(3-((5-chloro-4-(6-methyl-1H-indol-3-yl) pyrimidin-2-yl) amino)-5-cyclo propylbenzyl)-2,6-dimethylpiperazin-1-yl) ethan-1-ol;
6) (R)-5-chloro-N-(3-cyclopropyl-5-((3-methylpiperazin-1-yl) methyl) phenyl)-4-(1H-indol-3-yl) pyrimidine-2-amine;
7) (R)-5-chloro-N-(3-cyclopropyl-5-((3-methylpiperazin-1-yl) methyl) phenyl)-4-(6-methyl-1H-indol-3-yl) pyrimidin-2-amine;
8) 5-chloro-N-(3-cyclopropyl-5-(((3R,5S)-3,5-dimethylpiperazin-1-yl) methyl) phenyl)-4-(6-methyl-1H-indol-3-yl) pyrimidin-2-amine;
9) 5-chloro-N-(3-cyclopropyl-5-(((3S,5R)-3-ethyl-5-methylpiperazine-1-yl) methyl) phenyl)-4-(6-methyl-1H-indol-3-yl) pyrimidin-2-amine;
10) 5-chloro-N-(3-cyclopropyl-5-((3,5-dimethylpiperazin-1-yl) methyl) phenyl)-4-(6-methyl-1H-indol-3-yl) pyrimidin-2-amine;
11) N-(3-cyclopropyl-5-(((3R,5S)-3,5-dimethylpiperazin-1-yl) methyl) phenyl)-4-(6-methyl-1H-indol-3-yl) pyrimidin-2-amine;
12) N-(3-cyclopropyl-5-(((3R,5S)-3,5-dimethylpiperazin-1-yl) methyl) phenyl)-5-fluoro-4-(6-methyl-1H-indol-3-yl) pyrimidin-2-amine;
13) N-(3-cyclopropyl-5-(((3R,5S)-3,5-dimethylpiperazin-1-yl) methyl) phenyl)-4-(1H-indol-3-yl)-5-methyl pyrimidin-2-amine;
14) N-(3-cyclopropyl-5-(((3R,5S)-3,5-dimethylpiperazin-1-yl) methyl) phenyl)-5-methyl-4-(6-methyl-1H-indol-3-yl) pyrimidin-2-amine;
15) N-(3-cyclopropyl-5-(((3R,5S)-3,5-dimethylpiperazin-1-yl) methyl) phenyl)-4-(6-methyl-1H-indol-3-yl)-5-(trifluoromethyl) pyrimidin-2-amine;
16) 3-(5-chloro-2-((3-cyclopropyl-5-(((3R,5S)-3,5-dimethylpiperazine-1-yl) methyl) phenyl) amino) pyrimidin-4-yl)-1H-indol-6-yl) methanol;
17) 5-chloro-N-(3-cyclopropyl-5-(((3R,5S)-3,5-dimethylpiperazin-1-yl) methyl) phenyl)-4-(5-methoxy-6-methyl-1H-indol-3-yl) pyrimidin-2-amine;
18) 3-(5-chloro-2-((3-cyclopropyl-5-(((3R,5S)-3,5-dimethylpiperazin-1-yl) methyl) phenyl) amino) pyrimidin-4-yl)-6-methyl-1H-indol-5-ol;
19) 3-(5-chloro-2-((3-cyclopropyl-5-(((3R,5S)-3,5-dimethylpiperazin-1-yl) methyl) phenyl) amino) pyrimidin-4-yl)-6-methylindolin-2-on;
20) 5-chloro-N-(3-cyclopropyl-5-(((3R,5S)-3,5-dimethylpiperazin-1-yl) methyl) phenyl)-4-methoxy-6-(6-methyl-1H-indol-3-yl) pyrimidin-2-amine;
21) 5-chloro-2-((3-cyclopropyl-5-(((3R,5S)-3,5-dimethylpiperazin-1-yl) methyl) phenyl) amino)-6-(6-methyl-1H-indol-3-yl) pyrimidin-4-ol;
22) 3-(5-chloro-2-((3-cyclopropyl-5-(((3R,5S)-3,5-dimethylpiperazin-1-yl) methyl) phenyl) amino) pyrimidin-4-yl)-6-methyl-1H-indol-7-ol;
23) 2-((5-chloro-4-(6-methyl-1H-indol-3-yl) pyrimidin-2-yl) amino)-4-cyclopropyl-6-(((3R,5S)-3,5-dimethylpiperazin-1-yl) methyl) phenol;
24) 4-((5-chloro-4-(6-methyl-1H-indol-3-yl) pyrimidin-2-yl) amino)-2-cyclopropy-6-(((3R,5S)-3,5-dimethylpiperazin-1-yl) methyl) phenol;
25) (R)-5-chloro-N-(3-cyclopropyl-5-((3,3,5-trimethylpiperazin-1-yl) methyl) phenyl)-4-(6-methyl-1H-indol-3-yl) pyrimidin-2-amine;
26) ((2R,6R)-4-(3-((5-chloro-4-(6-methyl-1H-indol-3-yl) pyrimidin-2-yl) amino)-5-cyclopropylbenzyl)-6-methylpiperazin-2-yl) methanol;
27) (R)-5-chloro-N-(3-cyclopropyl-5-((5-methyl-4,7-diazaspiro [2.5]oxtan-7-yl) methyl) phenyl)-4-(6-methyl-1H-indol-3-yl) pyrimidin-2-amine;
28) 5-chloro-N-(3-cyclopropyl-5-(((3R,5R)-3,5-dimethylpiperazin-1-yl) methyl) phenyl)-4-(6-methyl-1H-indol-3-yl) pyrimidin-2-amine;
29) 5-chloro-N-(3-cyclopropyl-5-(((3S,5S)-3,5-dimethylpiperazin-1-yl) methyl) phenyl)-4-(6-methyl-1H-indol-3-yl) pyrimidin-2-amine;
30) 5-chloro-N-(3-cyclopropyl-5-(((3R,5S)-3,4,5-trimethylpiperazin-1-yl) methyl) phenyl)-4-(6-methyl-1H-indol-3-yl) pyrimidin-2-amine;
31) (2R,6S)-4-(3-((5-chloro-4-(6-methyl-1H-indol-3-yl) pyrimidin-2-yl) amino)-5-cyclopropylbenzyl)-2,6-dimethylpiperazin-1-ol; and
32) (2R,6S)-4-(3-cyclopropyl-5-((4-(6-methyl-1H-indol-3-yl) pyrimidine-2-yl) amino) benzyl)-2,6-dimethylpiperazin-1-ol.

20. The method of claim 1, wherein the acute myeloid leukemia is an acute myeloid leukemia having a FLT3 mutation.

21. The method of claim 1, wherein the acute myeloid leukemia is a mutant FLT3 polynucleotide-positive acute myeloid leukemia, an internal tandem duplication (ITD) in the FLT3 gene-positive acute myeloid leukemia, or an acute myeloid leukemia having a FLT3 point mutation.

22. The method of claim 1, wherein the acute myeloid leukemia has a tyrosine kinase domain (TKD) (FLT3-TKD) mutation of the FLT3 amino acid sequence.

23. The method of claim 1, wherein the FLT3-TKD mutation further comprises internal tandem duplication (ITD).

24. The method of claim 1, wherein the FLT3-TKD mutation includes a mutation selected from the group consisting of FLT3 (D835Y), FLT3 (F691L), FLT3 (F691L/D835Y), FLT3 (ITD/D835Y), FLT3 (ITD/F691L), and combinations thereof.

25. A pharmaceutical combination comprising a therapeutically effective amount of a compound of Chemical Formula 1, or a pharmaceutically acceptable salt thereof, solvate thereof, stereoisomer thereof, tautomer thereof, or combination thereof and (i) a B-cell lymphoma-2 (Bcl-2) inhibitor or (ii) Bcl-2 inhibitor and a hypomethylating agent (HMA); 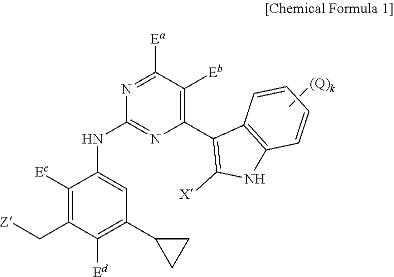
wherein in Chemical Formula 1,
Ea is hydrogen, hydroxy or C1-4 alkoxy;
Eb is hydrogen, halogen, C1-4 alkyl or C1-4 fluoroalkyl;
Ec and Ed are each independently hydrogen or hydroxy;
X′ is hydrogen or hydroxy;
k is an integer from 1 to 2;
each Q is independently of each other hydroxy, halogen, C1-4 alkyl, hydroxy C1-4 alkyl or C1-4 alkoxy;
Z′ is a monovalent functional group represented by Chemical Formula 2; 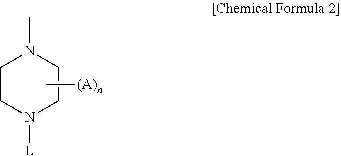
wherein in Chemical Formula 2,
each A is a functional group selected from hydroxy, C1-4 alkyl and hydroxy C1-4 alkyl, independently of each other, wherein at least one A is C1-4 alkyl;
n is an integer from 1 to 2; and
L is hydrogen, C1-4 alkyl, hydroxy or hydroxy C1-4 alkyl.

26. The pharmaceutical combination of claim 25, wherein the compound of Chemical Formula 1 is a Compound of Formula 3, or a pharmaceutically acceptable salt thereof, solvate thereof, stereoisomer thereof, tautomer thereof, or combination thereof; 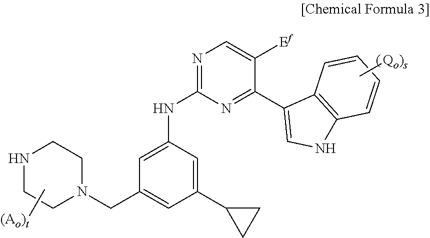
wherein in Chemical Formula 3,
Ef is fluorine, chlorine, bromine or iodine;
Qo is hydroxy, halogen, C1-4 alkyl, hydroxy C1-4 alkyl or C1-4 alkoxy;
s is an integer from 1 to 2;
Ao is a functional group selected from hydroxy, C1-4 alkyl and hydroxy C1-4 alkyl; and
t is an integer from 1 to 2.

27. The pharmaceutical combination of claim 25, wherein the compound of Chemical Formula 1 is the compound: 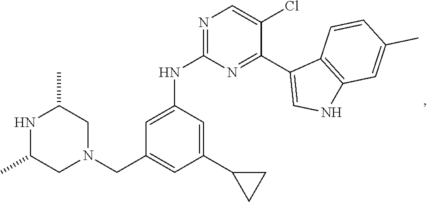
or a pharmaceutically acceptable salt thereof, solvate thereof, stereoisomer thereof, tautomer thereof, or combination thereof.

28. The pharmaceutical combination of claim 25, wherein the Bcl-2 inhibitor is selected from the group consisting of venetoclax, navitoclax, obatoclax, and combinations thereof.

29. The pharmaceutical combination of claim 25, wherein the hypomethylating agent (HMA) is selected from the group consisting of azacitidine, decitabine, idarubicin, and combinations thereof

30. The pharmaceutical combination of claim 25, wherein the compound of Chemical Formula 1 is administered in combination with a Bcl-2 inhibitor.

31. The pharmaceutical combination of claim 25, wherein the compound of Chemical Formula 1 is administered in combination with a Bcl-2 inhibitor and a hypomethylating agent (HMA).

32. The pharmaceutical combination of claim 25, wherein the compound of Chemical Formula 1 is administered in combination with venetoclax.

33. The pharmaceutical combination of claim 26, wherein the compound of Chemical Formula 3 is administered in combination with venetoclax.

34. The pharmaceutical combination of claim 27, wherein the compound is administered in combination with venetoclax.

35. The pharmaceutical combination of claim 25, wherein the compound of Chemical Formula 1 is administered in combination with venetoclax and at least one hypomethylating agent selected from azacitidine, decitabine, and idarubicin.

36. The pharmaceutical combination of claim 26, wherein the compound of Chemical Formula 3 is administered in combination with venetoclax and at least one hypomethylating agent selected from azacitidine, decitabine, and idarubicin.

37. The pharmaceutical combination of claim 27, wherein the compound is administered in combination with venetoclax and at least one hypomethylating agent selected from azacitidine, decitabine, and idarubicin.

38. The pharmaceutical combination of claim 27, wherein the compound is administered in combination with venetoclax and azacitidine.

39. The pharmaceutical combination of claim 25, wherein the (i) the Bcl-2 inhibitor, or (ii) the Bcl-2 inhibitor and the hypomethylating agent is:
(i) administered concurrently with the compound of Chemical Formula 1;
(ii) administered sequentially after the compound of Chemical Formula 1 is administered first;
(iii) administered first, before sequential administration of the compound of Chemical Formula 1; or
(iv) in a form in which the Bcl-2 inhibitor or the Bcl-2 inhibitor and the hypomethylating agent are administered, separately from the FLT3 inhibitor, in any order.

40. The pharmaceutical combination of claim 25, wherein the compound of Chemical Formula 1 and the Bcl-2 inhibitor or Bcl-2 inhibitor and a hypomethylating agent are co-formulated.

41. The pharmaceutical combination of claim 25, wherein the compound of Chemical Formula 1 and the Bcl-2 inhibitor or Bcl-2 inhibitor and a hypomethylating agent are formulated in separate dosage forms.

42. The pharmaceutical combination of claim 25, wherein the Bcl-2 inhibitor, or the Bcl-2 inhibitor and the hypomethylating agent and the compound of Chemical Formula 1 are administered, respectively,
(a) in a form in which at least one is administered orally, or
(b) in a form in which at least one is administered parenterally.

43. The pharmaceutical combination of claim 25, wherein the compound of Chemical Formula 1 is selected from the group consisting of:
1) 5-chloro-N-(3-cyclopropyl-5-(((3R,5S)-3,5-dimethylpiperazin-1-yl) methyl) phenyl)-4-(6-fluoro-1H-indol-3-yl) pyrimidin-2-amine;
2) 5-chloro-4-(6-chloro-1H-indol-3-yl)-N-(3-cyclopropyl-5-(((3R,5S)-3,
5-dimethylpiperazine-1-yl) methyl) phenyl) pyrimidin-2-amine;
3) 2-((2R,6S)-4-(3-((5-chloro-4-(6-fluoro-1H-indol-3-yl) pyrimidine-2-yl) amino)-5-cyclopropylbenzyl)-2,6-dimethylpiperazine-1-yl) ethan-1-ol;
4) 2-((2R,6S)-4-(3-((5-chloro-4-(1H-indol-3-yl) pyrimidine-2-yl) amino)-5-cyclopropylbenzyl)-2,6-dimethylpiperazine-1-yl) ethan-1-ol;
5) 2-((2R,6S)-4-(3-((5-chloro-4-(6-methyl-1H-indol-3-yl) pyrimidin-2-yl) amino)-5-cyclo propylbenzyl)-2,6-dimethylpiperazin-1-yl) ethan-1-ol;
6) (R)-5-chloro-N-(3-cyclopropyl-5-((3-methylpiperazin-1-yl) methyl) phenyl)-4-(1H-indol-3-yl) pyrimidine-2-amine;
7) (R)-5-chloro-N-(3-cyclopropyl-5-((3-methylpiperazin-1-yl) methyl) phenyl)-4-(6-methyl-1H-indol-3-yl) pyrimidin-2-amine;
8) 5-chloro-N-(3-cyclopropyl-5-(((3R,5S)-3,5-dimethylpiperazin-1-yl) methyl) phenyl)-4-(6-methyl-1H-indol-3-yl) pyrimidin-2-amine;
9) 5-chloro-N-(3-cyclopropyl-5-(((3S,5R)-3-ethyl-5-methylpiperazine-1-yl) methyl) phenyl)-4-(6-methyl-1H-indol-3-yl) pyrimidin-2-amine;
10) 5-chloro-N-(3-cyclopropyl-5-((3,5-dimethylpiperazin-1-yl) methyl) phenyl)-4-(6-methyl-1H-indol-3-yl) pyrimidin-2-amine;
11) N-(3-cyclopropyl-5-(((3R,5S)-3,5-dimethylpiperazin-1-yl) methyl) phenyl)-4-(6-methyl-1H-indol-3-yl) pyrimidin-2-amine;
12) N-(3-cyclopropyl-5-(((3R,5S)-3,5-dimethylpiperazin-1-yl) methyl) phenyl)-5-fluoro-4-(6-methyl-1H-indol-3-yl) pyrimidin-2-amine;
13) N-(3-cyclopropyl-5-(((3R,5S)-3,5-dimethylpiperazin-1-yl) methyl) phenyl)-4-(1H-indol-3-yl)-5-methyl pyrimidin-2-amine;
14) N-(3-cyclopropyl-5-(((3R,5S)-3,5-dimethylpiperazin-1-yl) methyl) phenyl)-5-methyl-4-(6-methyl-1H-indol-3-yl) pyrimidin-2-amine;
15) N-(3-cyclopropyl-5-(((3R,5S)-3,5-dimethylpiperazin-1-yl) methyl) phenyl)-4-(6-methyl-1H-indol-3-yl)-5-(trifluoromethyl) pyrimidin-2-amine;
16) 3-(5-chloro-2-((3-cyclopropyl-5-(((3R,5S)-3,5-dimethylpiperazine-1-yl) methyl) phenyl) amino) pyrimidin-4-yl)-1H-indol-6-yl) methanol;
17) 5-chloro-N-(3-cyclopropyl-5-(((3R,5S)-3,5-dimethylpiperazin-1-yl) methyl) phenyl)-4-(5-methoxy-6-methyl-1H-indol-3-yl) pyrimidin-2-amine;
18) 3-(5-chloro-2-((3-cyclopropyl-5-(((3R,5S)-3,5-dimethylpiperazin-1-yl) methyl) phenyl) amino) pyrimidin-4-yl)-6-methyl-1H-indol-5-ol;
19) 3-(5-chloro-2-((3-cyclopropyl-5-(((3R,5S)-3,5-dimethylpiperazin-1-yl) methyl) phenyl) amino) pyrimidin-4-yl)-6-methylindolin-2-on;
20) 5-chloro-N-(3-cyclopropyl-5-(((3R,5S)-3,5-dimethylpiperazin-1-yl) methyl) phenyl)-4-methoxy-6-(6-methyl-1H-indol-3-yl) pyrimidin-2-amine;
21) 5-chloro-2-((3-cyclopropyl-5-(((3R,5S)-3,5-dimethylpiperazin-1-yl) methyl) phenyl) amino)-6-(6-methyl-1H-indol-3-yl) pyrimidin-4-ol;
22) 3-(5-chloro-2-((3-cyclopropyl-5-(((3R,5S)-3,5-dimethylpiperazin-1-yl) methyl) phenyl) amino) pyrimidin-4-yl)-6-methyl-1H-indol-7-ol;
23) 2-((5-chloro-4-(6-methyl-1H-indol-3-yl) pyrimidin-2-yl) amino)-4-cyclopropyl-6-(((3R,5S)-3,5-dimethylpiperazin-1-yl) methyl) phenol;
24) 4-((5-chloro-4-(6-methyl-1H-indol-3-yl) pyrimidin-2-yl) amino)-2-cyclopropy-6-(((3R,5S)-3,5-dimethylpiperazin-1-yl) methyl) phenol;
25) (R)-5-chloro-N-(3-cyclopropyl-5-((3,3,5-trimethylpiperazin-1-yl) methyl) phenyl)-4-(6-methyl-1H-indol-3-yl) pyrimidin-2-amine;
26) ((2R,6R)-4-(3-((5-chloro-4-(6-methyl-1H-indol-3-yl) pyrimidin-2-yl) amino)-5-cyclopropylbenzyl)-6-methylpiperazin-2-yl) methanol;
27) (R)-5-chloro-N-(3-cyclopropyl-5-((5-methyl-4,7-diazaspiro [2.5]oxtan-7-yl) methyl) phenyl)-4-(6-methyl-1H-indol-3-yl) pyrimidin-2-amine;
28) 5-chloro-N-(3-cyclopropyl-5-(((3R,5R)-3,5-dimethylpiperazin-1-yl) methyl) phenyl)-4-(6-methyl-1H-indol-3-yl) pyrimidin-2-amine;
29) 5-chloro-N-(3-cyclopropyl-5-(((3S,5S)-3,5-dimethylpiperazin-1-yl) methyl) phenyl)-4-(6-methyl-1H-indol-3-yl) pyrimidin-2-amine;
30) 5-chloro-N-(3-cyclopropyl-5-(((3R,5S)-3,4,5-trimethylpiperazin-1-yl) methyl) phenyl)-4-(6-methyl-1H-indol-3-yl) pyrimidin-2-amine;
31) (2R,6S)-4-(3-((5-chloro-4-(6-methyl-1H-indol-3-yl) pyrimidin-2-yl) amino)-5-cyclopropylbenzyl)-2,6-dimethylpiperazin-1-ol; and
32) (2R,6S)-4-(3-cyclopropyl-5-((4-(6-methyl-1H-indol-3-yl) pyrimidine-2-yl) amino) benzyl)-2,6-dimethylpiperazin-1-ol.
